SEQUENCE LISTING (5 sequences)

SEQ ID NO: 1 - protein, 652 aa, Mus musculus
MAEAPQVVETDPDFEPLPRQRSCTWPLPRPEFNQSNSTTSSPAPSGGAAANPDAAASLASASAVSTDFMSNLSLLEESEDFARAPGCVAVAAAAAASRGLCGDFQGPEAGCVHPAPPQPPPTGPLSQPPPVPPSAAAAAGPLAGQPRKTSSSRRNAWGNLSYADLITKAIESSAEKRLTLSQIYEWMVKSVPYFKDKGDSNSSAGWKNAIRRNLALHSKFIRVQNEGTGKSSWWMLNPEGGKSGKSPRRRAASMDNNSKFAKSRGRAAKKKASLQSGQEGPGDSPGSQFSKWPASPGSHSNDDFDNWSTFRPRTSSNASTISGRLSPIMTEQDDLGDGDVHSLVYPPSAAKMASTLPSLSEISNPENMENLLDNLNLLSSPTSLTVSTQSSPGSMMQQTPCYSFAPPNTSLNSPSPNYSKYTYGQSSMSPLPQMPMQTLQDSKSSYGGLNQYNCAPGLLKELLTSDSPPHNDIMSPVDPGVAQPNSRVLGQNVMMGPNSVMPAYGSQASHNKMMNPSSHTHPGHAQQTASVNGRTLPHVVNTMPHTSAMNRLTPVKTPLQVPLSHPMQMSALGSYSSVSSCNGYGRMGVLHQEKLPSDLDGMFIERLDCDMESIIRNDLMDGDTLDFNFDNVLPNQSFPHSVKTTTHSWVSG

SEQ ID NO: 2 - protein, 655 aa, Homo sapiens
MAEAPQVVEIDPDFEPLPRPRSCTWPLPRPEFSQSNSATSSPAPSGSAAANPDAAAGLPSASAAAVSADFMSNLSLLEESEDFPQAPGSVAAAVAAAAAAAATGGLCGDFQGPEAGCLHPAPPQPPPPGPLSQHPPVPPAAAGPLAGQPRKSSSSRRNAWGNLSYADLITKAIESSAEKRLTLSQIYEWMVKSVPYFKDKGDSNSSAGWKNAIRRNLALHSKFIRVQNEGTGKSSWWMLNPEGGKSGKSPRRRAASMDNNSKFAKSRSRAAKKKASLQSGQEGAGDSPGSQFSKWPASPGSHSNDDFDNWSTFRPRTSSNASTISGRLSPIMTEQDDLGEGDVHSMVYPPSAAKMASTLPSLSEISNPENMENLLDNLNLLSSPTSLTVSTQSSPGTMMQQTPCYSFAPPNTSLNSPSPNYQKYTYGQSSMSPLPQMPIQTLQDNKSSYGGMSQYNCAPGLLKELLTSDSPPHNDIMTPVDPGVAQPNSRVLGQNVMMGPNSVMSTYGSQASHNKMMNPSSHTHPGHAQQTSAVNGRPLPHTVSTMPHTSGMNRLTQVKTPVQVPLPHPMQMSALGGYSSVSSCNGYGRMGLLHQEKLPSDLDGMFIERLDCDMESIIRNDLMDGDTLDFNFDNVLPNQSFPHSVKTTTHSWVSG

SEQ ID NO: 3 - protein, 94 aa, Homo sapiens
MTNDVTWSEASSPDERTLTFAERWQLSSPDGVDTDDDLPKSRASKRTCGVNDDESPSKIFMVGESPQVSSRLQNLRLNNLIPRQLFKPTDNQET

SEQ ID NO: 4 - protein, 228 aa, Aequorea victoria
MVSKGEELFTGVVPILVELDGDVNGHKFSVRGEGEGDATNGKLTLKFICTTGKLPVPWPTLVTTFGYGVACFSRYPDHMKQHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSHQSALSKDPNEKRDHMVLLEFVTAA

SEQ ID NO: 5 - protein, 235 aa, Aequorea victoria
MVSKGEELIKENMHMKLYMEGTVNNHHFKCTSEGEGKPYEGTQTMRIKAVEGGPLPFAFDILATSFMYGSKTFINHTQGIPDFFKQSFPEGFTWERVTTYEDGGVLTATQDTSLQDGCLIYNVKIRGVNFPSNGPVMQKKTLGWEASTETLYPADGGLEGRADMALKLVGGGHLICNLKTTYRSKKPAKNLKMPGVYYVDRRLERIKEADKETYVEQHEVAVARYCDLPSKLGHR

(19) United States
(12) Patent Application Publication
(10) Pub. No.: US 2016/0290995 A1
(43) Pub. Date: Oct. 6, 2016

(54) METHODS OF SELECTING AKT AGONISTS OR ANTAGONISTS

(71) Applicants: Sean Gross, Portland, OR (US); Peter Rotwein, El Paso, TX (US)

(72) Inventors: Sean Gross, Portland, OR (US); Peter Rotwein, El Paso, TX (US)

(73) Assignee: OREGON HEALTH & SCIENCE UNIVERSITY, PORTLAND, OR (US)

(21) Appl. No.: 15/085,757

(22) Filed: Mar. 30, 2016

Related U.S. Application Data
(60) Provisional application No. 62/140,629, filed on Mar. 31, 2015.

(51) Int. Cl. G01N 33/50

(52) U.S. Cl. CPC G01N 33/5023; G01N 2333/91205

(57) ABSTRACT

Disclosed herein are methods of identifying a test compound as agonists or antagonists of Akt activity. The methods involve contacting the test compound with a cell that expresses a biosensor comprising a FOXO1 or HDHB polypeptide and a fluorescent protein and locating the biosensor within the cell. Locating the biosensor in the nucleus relative to the cytoplasm is an indication that the test compound has an effect upon Akt activity.

### ACKNOWLEDGEMENT OF GOVERNMENT SUPPORT

This invention was made with the support of the United States government under the terms of R01 DK042748 and T32 CA 06195, both of which were awarded by the National Institutes of Health. The United States government has certain rights to this invention.

### FIELD

Generally, the field is methods of selecting test compounds. More specifically, the field is methods of selecting test compounds that inhibit or promote the activity of Akt.

### BACKGROUND

Cells respond to their environment through the actions of intracellular signaling pathways. An environmental agent, such as a peptide hormone or growth factor, typically binds to the extracellular surface of its trans-membrane receptor. Through changes in conformational energy, ligand binding triggers enzymatic activity in the intracellular part of the receptor, leading to production of short-lived second messengers and transient protein-protein interactions that activate multiple signaling networks. Despite many advances in biochemistry that have identified and characterized the components of these networks in intimate detail, knowledge of the dynamics of cellular signaling is limited. Studying individual responses within a population has been particularly challenging because most experimental methods lack sufficient sensitivity, or exhibit low temporal or spatial resolution. Moreover, signaling pathways do not function in isolation but may be interconnected, non-linear, or contain a variety of feedback and feed-forward modifiers that complicate analyses (Purvis J E and Lahav G Cell 152, 945-956 (2013); incorporated by reference herein)

Live cell imaging using a sensitive, specific, and quantifiable sensor resolves several of the limitations inherent in biochemical assays. By allowing many individual cells within a population to be tracked with high temporal and spatial fidelity, this approach can result in major improvements in both the amount and quality of acquired data, often leading to surprising new insights (Purvis and Lahav, 2013 supra). For example, responses to signals activating the transcription factor NFκβ were shown to be digital, in the sense that individual cells either did or did not respond to a given stimulus (Tay S et al, Nature 466, 267-271 (2010); incorporated by reference herein). Responding cells also exhibited pulsatile behavior, typically showing several peaks of activity that were asynchronous within the population (Tay et al, 2010 supra). Similar complex signaling dynamics have been found in the Erk kinase pathway, where responses were asynchronous and pulsatile in MCF-10 mammary epithelial cells exposed to epidermal growth factor (EGF), with the amplitude and duration of pulses dependent on EGF concentrations (Albeck J G et al, Mol Cell 49, 249-261 (2013); incorporated by reference herein).

The three highly-related mammalian Akt protein kinases are activated by hormones and growth factors that stimulate class Ia PI3-kinases to produce the signaling intermediate, PIP3 (phosphatidyl-inositol 3,4,5 trisphosphate) (Manning and Cantley, 2007 infra). PIP3 targets Akt to the inner face of the cell membrane by association with its pleckstrin-homology domain, leading to Akt activation via sequential phosphorylation by upstream kinases PDK-1 and mTorc2 (Hay, 2011 infra; Toker, 2012 infra). Once stimulated, Akt can phosphorylate many substrates within several subcellular compartments (Hay N, Biochim Biophys Acta 1813, 1965-1970 (2011); Manning B D and Cantley L C, Cell 129, 1261-12174 (2007); Toker A, Adv Biol Regul 52, 78-87 (2012); all of which are incorporated by reference herein). These substrate proteins include mediators of immediate changes in cell shape, movement, and intermediary metabolism, or are components of longer-term effects on cell viability, division, or differentiation (Hay, 2011 supra; Manning and Cantley, 2007 supra; Toker, 2012 supra).

Readouts to assess Akt activity in living cells are limited. Studies using different FRET-based reporters have been published, but they tend to suffer from low signal-to-noise ratios, and exhibit poor off-rate kinetics (Gao X and Zhang J, Mol Biol Cell 19, 4366-4373 (2008); Komatsu N K et al, Mol Biol Cell 22, 4647-4656 (2011); Kunkel M T et al, J Biol Chem 280, 5581-5587 (2005); Miura H et al, Cell Struct Funct 39, 9-20 (2014); Sasaki K et al, J Biol Chem 278, 30945-30951 (2003); Yoshizaki H et al, Mol Biol Cell 18, 119-128 (2007); Zhang L et al, Nat Med 13 1114-1119, 2007); all of which are incorporated by reference herein). In addition, the complex equipment and expertise needed to measure and quantify FRET has prevented these systems from being widely adopted. An alternative approach has been developed using an Akt-based fluorescent fusion protein (Meyer R et al, Front Physiol 3, 451 (2012); incorporated by reference herein), but the application of this reagent to quantify single cell responses has been problematic because of measurement difficulties related to repeatedly imaging a small segment of the cell membrane.

Clearly, a method of assessing Akt activity in living cells at the level of a single cell is a necessary development for understanding Akt biology and for selecting test compounds for inhibition or activation of Akt.

### SUMMARY

Disclosed herein are methods of identifying test compounds as agonists or antagonists of Akt activity. One such method that identifies agonists of Akt activity involves providing a first Akt expressing cell, the first Akt expressing cell comprising a biosensor, the biosensor comprising a first polypeptide of SEQ ID NO: 1, SEQ ID NO: 2, SEQ ID NO: 3 or any polypeptide with at least 95% amino acid identity to those sequences provided that a biosensor comprising such a polypeptide has equivalent activity to a biosensor comprising SEQ ID NO: 1, SEQ ID NO: 2, or SEQ ID NO: 3. The first Akt expressing cell is provided in a media that does not activate Akt, such as a serum free media. The method further involves providing a second Akt expressing cell. The second Akt expressing cell is provided in the same media as the first Akt expressing cell and includes the same biosensor. The first Akt expressing cell is contacted with a first composition. The first composition includes a first test compound at a first concentration and a vehicle. The second Akt expressing cell is contacted with a second composition. The second composition is made up of vehicle alone. This second Akt expressing cell serves as a negative control. The method further involves measuring the relative nuclear intensity of the fluorescent protein over time in the first Akt expressing cell and in the second Akt expressing cell. A higher rate of decrease of the relative nuclear intensity of the fluorescent protein in the first Akt expressing cell relative to that of the negative control is an indication that the test compound is an agonist of Akt activity.

Another such method that identifies antagonists of Akt activity involves providing a first Akt expressing cell. The first Akt expressing cell includes an expression vector. The expression vector includes a first polynucleotide that encodes a biosensor comprising SEQ ID NO: 1, SEQ ID NO: 2, SEQ ID NO: 3, or any polypeptide with at least 95% amino acid identity to those sequences provided that a biosensor comprising such a polypeptide has equivalent activity to a biosensor comprising SEQ ID NO: 1, SEQ ID NO: 2, or SEQ ID NO: 3. The first Akt expressing cell is provided in a media comprising a compound known to activate Akt, such as IGF-1, fetal bovine serum, insulin, or PDGF-BB. The method further involves providing a second Akt expressing cell. The second Akt expressing cell is provided in the same media as the first Akt expressing cell and includes the same biosensor. The first Akt expressing cell is contacted with a first composition. The first composition includes a first test compound at a first concentration and a vehicle. The second Akt expressing cell is contacted with a second composition. The second composition is made up of vehicle alone. This second Akt expressing cell serves as a negative control. The method further involves measuring the relative nuclear intensity of the fluorescent protein over time in the first Akt expressing cell and in the second Akt expressing cell over time. A higher rate of increase of the relative nuclear intensity of the fluorescent protein in the first Akt expressing cell relative to that of the negative control is an indication that the test compound is an antagonist of Akt activity.

For the above methods, the fluorescent protein can be any fluorescent protein, including Clover fluorescent protein (SEQ ID NO: 4) or the mKate fluorescent protein (SEQ ID NO: 5). Compositions comprising the test compound can include different concentrations of test compounds and applied to other Akt expressing cells that also include the biosensor and a dose-response to the test compound calculated. The test compound can be any test compound such as a protein, antibody, or small molecule. The Akt expressing cells can express Akt endogenously or exogenously (for example, if the cell includes an expression vector that drives the expression of Akt). The methods can further comprise measuring the relative cytoplasmic activity of the fluorescent protein over time. The Akt expressing cell can comprise an expression vector comprising a polynucleotide that encodes the biosensor and a promoter operably linked to the polynucleotide. Relative nuclear intensity and relative cytoplasmic intensity can be measured by any method including live cell imaging.

Also disclosed are recombinant biosensors comprising a FOXO1 domain of SEQ ID NO: 1 or SEQ ID NO: 2 and a fluorescent protein N or C terminal to the FOXO1 domain. The fluorescent protein can be any such protein including clover (SEQ ID NO: 4) or mKate (SEQ ID NO: 5).

### BRIEF DESCRIPTION OF THE SEVERAL VIEWS OF THE DRAWINGS

Some of the drawings herein are best understood in color. Applicants consider the original color versions of the drawings herein part of the original disclosure and reserve the right to submit the color versions of the drawings in later proceedings.

FIG. 1A is a schematic of FOXO1-clover reporter protein showing locations of three Akt phosphorylation sites (T24, S256, and S319) and three amino acid substitutions engineered into the Forkhead DNA binding domain (FKH) (S212A, H215R, and S218A) (numbering equivalent to that of human FOXO1.) Also indicated are locations of the nuclear localization sequence (NLS) and nuclear export sequence (NES) of FOXO1; FP, fluorescent protein.

FIG. 1B is a diagram of the expected location of the FOXO1-clover reporter in cells with low Akt activity, where FOXO1 is not phosphorylated (P) and is predominantly nuclear, or high activity, where FOXO1 is highly phosphorylated and is primarily cytoplasmic.

FIG. 1C is a set of time-lapse images of a representative experiment showing changes in the subcellular location of the FOXO1-clover reporter in 10T1/2 cells exposed to R3-IGF-I [250 pM] for the times indicated vs. continual incubation in serum-free medium (SFM). Scale bar=50 μM.

FIG. 2A is plot of the results of live tracking of 16 individual cells incubated in 10% FBS for 12 hours, starting after mitosis and followed by incubation for 90 min in SFM. The relative nuclear intensity of the FOXO1-clover reporter protein recorded on the graph has been normalized to the average value at 90 min after addition of SFM.

FIG. 2B is a heat map showing the relative nuclear intensity of the reporter protein in each of 122 individual cells analyzed for up to 16 hours in 10% FBS followed by 90 min in SFM. Nuclear intensity values for each cell were normalized to the value 90 min after addition of SFM (labeled red for high relative nuclear reporter localization). Cells have been aligned computationally beginning with the time since mitosis.

FIG. 3A is a plot showing a time course of relative nuclear intensity of the FOXO1-clover reporter in cells incubated in SFM and then exposed to SFM, BMP-2 [15 nM], R3-IGF-I [1 nM], 10% FBS, or PDGF-BB [206 pM] for 60 min. Population averages are presented (n=50 cells per incubation). The nuclear intensity of the reporter in each cell was normalized to its value at the start of imaging during incubation in SFM.

FIG. 3B is an image of an immunoblot showing expression of phosphorylated Akt (pAkt^{T308}), total Akt, pSmad5, total Smad, and α-tubulin by immunoblotting using whole cell protein lysates from the same population analyzed in FIG. 3A after exposure to the indicated growth factors or SFM for 60 min. The 50 kDa molecular mass marker is indicated to the right of each immunoblot.

FIG. 4A is a plot showing a time course of relative nuclear intensity of the FOXO1-clover reporter in 10T1/2 cells incubated in SFM and then exposed to different concentrations of R3-IGF-I as indicated for 60 min. Population averages are presented (n=50 cells per incubation).

FIG. 4B is an image of an immunoblot showing Expression of phosphorylated Akt (pAkt^{T308}) and total Akt by immunoblotting using whole cell protein lysates from the same population analyzed in FIG. 4A.

FIG. 4C is a plot showing a time course of relative nuclear intensity of the FOXO1-clover reporter in C2 myoblasts incubated in SFM and then exposed to different concentrations of R3-IGF-I as indicated for 60 min. Population averages are presented (n=50 cells per incubation).

FIG. 4D is an image of an immunoblot showing Expression of pAkt and total Akt by immunoblotting using whole cell protein lysates from the same population analyzed in FIG. 4C.

Cells were imaged every 2 min in FIG. 4A and FIG. 4C, and the nuclear intensity of the reporter in each cell was normalized to its value at the start of imaging during incubation in SFM. Arrows in FIG. 4B and FIG. 4D represent the location of the 50 kDa molecular mass marker.

FIG. 5A is a plot showing time course results for each of 25 10T1/2 cells incubated with 50 pM R3-IGF-I for 60 min.

FIG. 5B is a plot showing time course results for each of 25 10T1/2 cells incubated with 500 pM R3-IGF-I for 60 min.

FIG. 5C is a plot showing time course results for each of 25 C2 myoblasts incubated with 12.5 pM R3-IGF-I for 60 min.

FIG. 5D is a plot showing time course results for each of 25 C2 myoblasts incubated with 125 pM R3-IGF-I for 60 min.

For all of FIG. 5A-FIG. 5D, cells were imaged every two minutes.

FIG. 5E is a set of histograms of individual 10T1/2 cells exposed to SFM or to different concentrations of R3-IGF-I for 60 min showing the frequency of the final relative nuclear localization values (^{˜}200 cells per each treatment).

FIG. 5F is a set of histograms of individual C2 myoblasts exposed to SFM or to different concentrations of R3-IGF-I for 60 min showing the frequency of the final relative nuclear localization values (^{˜}200 cells per each treatment).

FIG. 6A is a plot of a time course of relative nuclear intensity of the FOXO1-clover reporter in 10T1/2 cells incubated with SFM (navy tracing), sequentially with two exposures to R3-IGF-I ([500 pM], blue tracing; [50 pM], red tracing]), or with SFM followed by R3-IGF-I ([50 pM], green tracing). Population averages are presented (n=50 cells per incubation).

FIG. 6B is a plot of time course results for each of 25 individual cells incubated with 50 pM R3-IGF-I.

FIG. 6C is a plot of time course results for each of 25 individual cells incubated with 500 pM R3-IGF-I.

FIG. 7A is a plot of a time course of relative nuclear intensity of the FOXO1-clover reporter in 10T1/2 cells incubated in different concentrations of R3-IGF-I as indicated for 60 min, followed by addition of leptomycin B ([100 nM], Lepto) alone or with PI103 [500 nM] for 180 min. Population averages are presented (n=50 cells per incubation). The arrow indicates the time of addition of Lepto/PI103. Cells were imaged every two minutes.

FIG. 7B is a plot of a time course of relative nuclear intensity of the FOXO1-clover reporter in each of 5 individual 10T1/2 cells pre-incubated with R3-IGF-I [250 pM] followed at time 0 by addition Lepto or Lepto plus PI103 as in FIG. 7A. Cells were imaged every two minutes.

FIG. 7C is a dot plot of the rate of nuclear import of the FOXO1-clover reporter in cells incubated with Lepto (blue) or Lepto plus PI103 (red), determined by fitting time course traces of individual cells to a single exponential equation (n=74 cells/treatment group). Mean population values are represented by a black bar (p<0.0001, unpaired t-test).

FIG. 8A is a plot of a time course of relative nuclear intensity of the FOXO1-clover reporter in 10T1/2 cells incubated in SFM for 60 min and then exposed sequentially to PI103 [500 nM] (red and blue tracings) and leptomycin B [100 nM] (blue tracing), as indicated by the vertical arrows. The green tracing represents cells incubated in SFM for the entire 180 min experimental period. Cells were imaged every two minutes.

FIG. 8B is a plot of a time course of relative nuclear intensity of the FOXO1-clover reporter in 10T1/2 cells incubated with R3-IGF-I [500 pM] for 60 min, followed by PI103 [500 nM] and leptomycin B [100 nM] [vertical arrows indicate time of additions] for 60 min each (green tracing). The blue tracing represents results of cells incubated with leptomycin B for 180 min. Cells were imaged every two minutes.

FIG. 8C is a plot of quantitative data from individual cells (n=25) plotted from the experiments depicted in the green tracing in FIG. 8B. The average ratio of nuclear to cytoplasmic fluorescence (N/C) is listed above each cluster of individual cells. See Materials and Methods for additional details.

FIG. 8D is a set of time-lapse images of a field of cells from the experiment graphed in FIG. 8B. Scale bar=50 μM.

FIG. 9A is a diagram showing an overview of growth factor signaling described in Example 6 herein. Binding of insulin, IGF-I, EGF, PDGF-AA, and PDGF-BB to their respective tyrosine kinase receptors. The locations of action of the small molecule tyrosine kinase inhibitors, Linisitib and Sunitinib, are indicated.

FIG. 9B is a diagram of the steps leading to the activation of Akt and its regulation of the FoxO1-clover reporter protein. The targets of various inhibitors are depicted. RTK—receptor tyrosine kinase, TKI—tyrosine kinase inhibitor, PI103—small molecule inhibitor of PI3-kinase (PI3K) and mTorc2.

FIG. 9C is a table showing the relative expression of the indicated receptor (R) mRNAs is using RNA seq data.

FIGS. 9D-9H collectively show graded responses of the FoxO1-clover reporter to different concentrations of insulin.

FIG. 9D is a plot of the time course of the relative nuclear intensity of the FoxO1-clover reporter in C3H10T1/2 cells first incubated in serum-free medium (SFM), and following exposure to different concentrations of insulin for 90 min. Population means are presented (n=100 cells per incubation from two independent experiments). The nuclear intensity of the reporter in each cell was normalized to its value at the start of imaging during incubation in SFM.

FIG. 9E is an image of an immunoblot showing the expression of phosphorylated Akt (pAktT308), total Akt, phosphorylated PRAS40 (pPRAS40T246), and total PRAS40 by immunoblotting using whole cell protein lysates from cells exposed to insulin [1400 pM] for up to 90 min. Molecular mass markers are indicated to the right of each immunoblot.

FIG. 9F is a plot of the relative nuclear intensity of the FoxO1-clover reporter in 25 individual C3H10T1/2 cells first incubated in serum-free medium (SFM) incubated with 170 pM insulin for 90 minutes.

FIG. 9G is a plot of the relative nuclear intensity of the FoxO1-clover reporter in 25 individual C3H10T1/2 cells first incubated in serum-free medium (SFM) incubated with 1400 pM insulin for 90 minutes.

FIG. 9H is a set of time-lapse images from a representative experiment showing changes in the subcellular location of the FoxO1-clover reporter in cells exposed to insulin [1400 pM] for the times indicated. Scale bars=50 μM.

FIG. 10A is a plot showing the time course of relative nuclear intensity of the FoxO1-clover reporter in C3H10T1/2 cells incubated first in SFM, and following exposure to different concentrations of EGF for 90 min. Population means are presented (n=100 cells per incubation from two independent experiments). The nuclear intensity of the reporter in each cell was normalized to its value at the start of imaging during incubation in SFM.

FIG. 10B is an image of an immunoblot showing expression of pAkt, total Akt, pPRAS40, and total PRAS40 using whole cell protein lysates from cells exposed to EGF [4.2 nM] for up to 90 min. Molecular mass markers are illustrated to the right of each immunoblot.

FIG. 10C is a time course of the relative nuclear intensity of the FoxO1-clover reporter in 25 individual C3H10T1/2 cells incubated first in SFM and then with 0.4 nM EGF for 90 minutes.

FIG. 10D is a time course of the relative nuclear intensity of the FoxO1-clover reporter in 25 individual C3H10T1/2 cells incubated first in SFM and then with 4.2 nM EGF for 90 minutes.

FIG. 10E is a set of time-lapse images from a representative experiment showing changes in the subcellular location of the FoxO1-clover reporter in cells exposed to EGF [4.2 nM] for the times indicated. Scale bars=50 μM.

FIG. 10F is a set of three histograms of individual cells exposed to different concentrations of EGF showing the frequency of the time to peak response (^{˜}100 cells per group). The terms ‘Low’, ‘Medium’, and ‘High’ refer to the level of peak EGF-mediated signaling activity.

FIG. 10G is a plot showing that repeated exposure to EGF yields reduced population responses. Time course of relative nuclear intensity of the FoxO1-clover reporter in C3H10T1/2 cells incubated with SFM (red tracing), with EGF [4.2 nM] (orange), sequentially with two exposures to EGF (aqua), with SFM followed by EGF (green), or with EGF followed by IGF-I [500 pM] (blue). Population averages are presented (n=50 cells per incubation).

FIG. 11A is a plot of a time course of relative nuclear intensity of the FoxO1-clover reporter in C3H10T1/2 cells incubated in SFM and followed by exposure to different concentrations of PDGF-AA for 90 min. Population means are presented (n=150 cells per incubation from three independent experiments). The nuclear intensity of the reporter in each cell was normalized to its value at the start of imaging during incubation in SFM.

FIG. 11B is an image of an immunoblot of expression of pAkt, total Akt, pPRAS40, and total PRAS40 using whole cell protein lysates from cells exposed to PDGFAA [1400 pM] for up to 90 min. Molecular mass markers are illustrated to the right of each immunoblot.

FIG. 11C is a plot of a time course of relative nuclear intensity of the FoxO1-clover reporter in 25 individual C3H10T1/2 cells incubated in SFM and followed by exposure to 140 pM PDGF-AA for 90 min.

FIG. 11D is a plot of a time course of relative nuclear intensity of the FoxO1-clover reporter in 25 individual C3H10T1/2 cells incubated in SFM and followed by exposure to 1400 pM PDGF-AA for 90 min.

FIG. 11E is a set of time-lapse images from a representative experiment showing changes in the subcellular location of the FoxO1-clover reporter in cells exposed to PDGF-AA [1400 pM] for the times indicated. Scale bars=50 μM.

FIG. 12A is a set of four plots of representative single cell traces based on the type of response seen after incubation with PDGF-AA: no response is illustrated by the graph with the red lines; small transient responses are shown in gold; larger transient responses are in green, and large sustained responses in blue. The black line in each graph shows the average response across that cluster.

FIG. 12B is a dot-plot illustrating the correlation of responses of individual cells after incubation with PDGF-AA at 18 and 90 min after growth factor addition (n=900 cells). Color-coding is 671 identical to that of FIG. 12A.

FIG. 12C is a bar graph showing the fraction of cells in the population responding to incubation with different concentrations of PDGF-AA. The color-coding is identical to FIG. 12A.

FIG. 13A is a plot of a time course of relative nuclear intensity of the FoxO1-clover reporter in C3H10T1/2 cells first incubated in SFM and then followed by exposure to different concentrations of PDGF-BB for 90 min. Population means are depicted (n=150 cells per incubation from three independent experiments). The nuclear intensity of the reporter in each cell was normalized to its value at the start of imaging during incubation in SFM (=100).

FIG. 13B is an image of an immunoblot of expression of pAkt, total Akt, pPRAS40, and total PRAS40 by immunoblotting using whole cell protein lysates from cells exposed to PDGF-BB [104 pM] for up to 90 min. Molecular mass markers are shown to the right of each immunoblot.

FIG. 13C is a plot of a time course of relative nuclear intensity of the FoxO1-clover reporter in 25 individual C3H10T1/2 cells first incubated in SFM and then followed by exposure to 5.2 pm of PDGF-BB for 90 min.

FIG. 13D is a plot of a time course of relative nuclear intensity of the FoxO1-clover reporter in 25 individual C3H10T1/2 cells first incubated in SFM and then followed by exposure to 104 pm of PDGF-BB for 90 min.

FIG. 13E is a set of time-lapse images from a representative experiment showing changes in the subcellular location of the FoxO1-clover reporter in cells exposed to PDGF-BB [104 pM] for the times indicated. Scale bars=50 μM.

FIG. 13F is a dot-plot illustrating the pattern of responses of individual cells after incubation with PDGF-BB at 18 and 90 min after growth factor addition (n=900 cells). Color coding is the same as that of FIG. 12A.

FIG. 13G is a bar graph showing the fraction of cells in the population responding to incubation with different concentrations of PDGF-BB. Color-coding is the same as that of FIG. 12A.

FIG. 14A is a plot of a time course of relative nuclear intensity of the FoxO1-clover reporter in C3H10T1/2 cells incubated in SFM and followed by exposure to PDGF-AA [1400 pM] for 90 min±either anti-PDGF-α receptor antibody (αPDGFRα) or IgG control. Population means are presented (n=50 cells). The nuclear intensity of the reporter in each cell 700 was normalized to its value at the start of imaging during incubation in SFM (=100).

FIG. 14B is a plot of a time course of relative nuclear intensity of the FoxO1-clover reporter in C3H10T1/2 cells incubated in SFM and followed by exposure to different concentrations of PDGF-BB for 90 min±αPDGFRα. Population means are presented (n=50 cells). The nuclear intensity of the reporter in each cell was normalized to its value at the start of imaging during incubation in SFM (=100).

FIG. 14C is a is a plot of a time course of relative nuclear intensity of the FoxO1-clover reporter in 25 individual C3H10T1/2 cells incubated in SFM and followed by exposure to PDGF-BB [10.4 pM] for 90 min. without anti-PDGF-α receptor antibody (αPDGFRα).

FIG. 14D is a is a plot of a time course of relative nuclear intensity of the FoxO1-clover reporter in 25 individual C3H10T1/2 cells incubated in SFM and followed by exposure to PDGF-BB [10.4 pM]+anti-PDGFRα for 90 min.

FIG. 15A is a plot of a time course of relative nuclear intensity of the FoxO1-clover reporter in C3H10T1/2 cells incubated with SFM or PDGF-BB [830 pM] for 90 min in the presence of various concentrations of the receptor tyrosine kinase inhibitor, Sunitinib. Population averages are presented (n=50 cells per incubation).

FIG. 15B is a plot of a time course of relative nuclear intensity of the FoxO1-clover reporter in cells incubated with SFM or PDGF-BB [830 pM] for 90 min in the presence of different concentrations of PI103. Population averages are presented (n=50 cells per incubation).

FIG. 15C is a plot of a time course of relative nuclear intensity of the FoxO1-clover reporter in cells incubated with SFM or IGF-I [500 pM] for 90 min in the presence of various concentrations of the receptor tyrosine kinase inhibitor, Linsitinib. Population averages are presented (n=50 cells per incubation).

FIG. 15D is a plot of a time course of relative nuclear intensity of the FoxO1-clover reporter in cells incubated with SFM or IGF-I [500 pM] for 90 min in the presence of different concentrations of PI103. Population averages are presented (n=50 cells per incubation).

FIG. 16A is a plot of a time course of relative nuclear intensity of the FoxO1-clover reporter in HeLa cells first incubated in SFM and then exposed to different growth factors for 90 min. Population means are depicted (n=50 cells per incubation). The nuclear intensity of the reporter in each cell was normalized to its value at the start of imaging during incubation in SFM (=100).

FIG. 16B is a plot of a time course of relative nuclear intensity of the FoxO1-clover reporter in 25 individual HeLa cells first incubated in SFM and then exposed to 500 pM IGF-I.

FIG. 16C is a plot of a time course of relative nuclear intensity of the FoxO1-clover reporter in 25 individual HeLa cells first incubated in SFM and then exposed to 4.1 nM PDGF-BB.

FIG. 16D is a plot of a time course of relative nuclear intensity of the FoxO1-clover reporter in 25 individual HeLa cells first incubated in SFM and then exposed to 4.2 nM EGF.

FIG. 16E is a set of time lapse images from a representative experiment showing changes in the subcellular location of the FoxO1-clover reporter in cells exposed to different growth factors for the times indicated. Scale bars=25 μM.

FIG. 17A Top: is a Schematic of mKate2-HDHB reporter protein showing locations of four CDK2 phosphorylation sites and the nuclear localization sequence (NLS) and nuclear export sequence (NES) of HDHB; FP, fluorescent protein. Bottom: Diagram of the expected location of the mKate2-HDHB reporter in cells with low cell cycle activity (e.g., during G1 phase), where HDHB is not phosphorylated (P) and is predominantly nuclear, or high activity (e.g., during S-G2 phases), where HDHB is highly phosphorylated and is primarily cytoplasmic.

FIG. 17B is an image from a representative experiment showing variation in the subcellular location of mKate-HDHB in C3H10T1/2 cells exposed to 10% FBS. Scale bar=50 μM.

FIG. 17C is a plot of single cell traces of relative mKate2-HDHB nuclear intensity tracked over the full cell cycle in cells grown in 10% FBS (n=25 cells). Cells were computationally synchronized based on their time of division. Black circles show the point in time when a cell exited G1.

FIG. 17D is a graph showing the calculated times of G1 (blue) and S-M (red) phase of the cell cycle for individual cells grown in 10% FBS (n=50).

FIG. 18A is a plot showing single cell traces of relative mKate2-HDHB nuclear intensity tracked for 24 hr in cells treated with serum free media at time 0 (n=25 cells). Black circles show the point in time when a cell exited G1.

FIG. 18B is a is a plot showing single cell traces of relative mKate2-HDHB nuclear intensity tracked for 24 hr in cells treated with 500 pM IGF-I at time 0 (n=25 cells). Black circles show the point in time when a cell exited G1.

FIG. 18C is a set of time-lapse images of a representative experiment showing changes in the subcellular location of mKate-HDHB and FoxO1-clover reporters in C3H10T1/2 cells exposed to R3-IGF-I [250 pM] for the times indicated. Scale bar=50 μM.

FIG. 19A is a plot of a time course of relative nuclear intensity of the FoxO1-clover reporter in C3H10T1/2 cells incubated with IGF-I [500 pM] for 24 hr in the presence of Linsitinib [500 nM] added at different times as indicated. Population averages are presented (n=150 cells per treatment group).

FIG. 19B is a bar graph showing the percent of the cell population that exited G1 during a 24-hr exposure to SFM, IGF-I [500 pM], plus different times of addition of Linsitinib [500 nM]. Population means [±s.e.] are plotted from 3 independent experiments (50 cells/treatment/experiment). See Methods for details.

FIG. 19C is a plot of the time to progression through the G1 phase of the cell cycle in individual cells incubated with IGF-I [500 pM] plus Linsitinib [500 nM] added at different times.

FIG. 19D is a plot of the Effect of Linsitinib on cell cycle progression. Each dot represents integrated Akt signaling activity in an individual cell (summed nuclear localization of the FoxO1-clover reporter) after 24-hr of treatment with IGF-I±addition of Linsitinib [500 nM] at different times. Cells have been separated into those that exited G1 during the tracking period (+, left side) from those remaining in G1 (−, right side), based on redistribution of the HDHB-mKate2 reporter molecule.

FIG. 20A is a plot of a time course of relative nuclear intensity of the FoxO1-clover reporter in C3H10T1/2 cells incubated with SFM or IGF-I [500 pM] for 24 hr in the presence of the indicated concentrations of the receptor tyrosine kinase inhibitor, Linsitinib. Population averages are presented (n=200 cells per treatment group).

FIG. 20B is a bar graph showing the percent of the cell population that exited G1 (see Methods for details) during a 24-hr exposure to SFM, IGF-I [500 pM], plus different concentrations of Linsitinib or IGF-I. Population means [±s.e.] are plotted from 4 independent experiments (50 cells/treatment/experiment).

FIG. 20C is a plot of the time to progression through the G1 phase of the cell cycle in individual cells incubated with SFM or IGF-I [500 pM]±different concentrations of Linsitinib.

FIG. 20D is a plot of the effect of Linsitinib on cell cycle progression. Each dot represents integrated Akt signaling activity in an individual cell (summed nuclear localization of the FoxO1-clover reporter) after 24-hr of treatment with SFM or IGF-I±different concentrations of Linsitinib. Cells have been separated into those that exited G1 during the tracking period (+, left side) from those remaining in G1 (−, right side), based on redistribution of the HDHB-mKate2 reporter molecule.

FIG. 21A is a time course of the relative nuclear intensity of the FoxO1-clover reporter in C3H10T1/2 cells incubated in SFM for 24 hr, and then exposed to SFM, EGF [4.2 nM], PDGF-AA [1.4 nM], PDGF-BB [1.4 nM], or R3-IGF-I [500 pM] for 24 hr. Population means are presented (n=150-200 cells per group). The nuclear intensity of the reporter in each cell was normalized to its value at the start of imaging.

FIG. 21B is a bar graph of the percentage of cells (mean±SEM; n=4 independent experiments) that exited G1 during incubation in different growth factors for 24 hr.

FIG. 21C is a plot of Integrated Akt signaling activity in individual cells measured during 24 hr of growth factor exposure using the nuclear intensity of the FoxO1-clover reporter protein as in FIG. 21A. Vertical lines separate cells that exited the G1 phase of the cell cycle (+, left side) from those remaining in G1 (−, right side), based on redistribution of the HDHB-mKate2 reporter molecule.

FIG. 21D is a plot of the time of G1 exit in individual cells incubated in different growth factors, as in FIG. 21B. Each black horizontal line represents the average time of G1 exit after initiation of growth factor treatment.

FIG. 22A is a plot of time course results for each of 25 cells incubated in SFM for 24 hr, and then tracked for an additional 24 hr.

FIG. 22B is a plot of time course results for each of 25 cells incubated in SFM for 24 hr, and tracked after addition of PDGF-BB [1.4 nM] for 24 hr.

FIG. 22C is a plot of time course results for each of 25 cells incubated in SFM for 24 hr, and tracked after incubation with IGF-I [500 pM] for an additional 24 hr.

FIG. 22D is a plot of time course results for each of 25 cells incubated in SFM for 24 hr, and tracked after addition of PDGF-AA [1.4 nM] for 24 hr.

FIG. 22E is a set of time-lapse images from a representative experiment showing changes in the subcellular location of the FoxO1-clover reporter in C310T1/2 cells after incubation with PDGF-AA [1.4 nM] for up to 15 hr. The red circle within each image identifies the same two cells, and illustrates the oscillations in the nuclear localization of the FoxO1-clover reporter protein. Scale bars=50 μM.

FIG. 23A is a plot showing the relative migration distance (microns) for 10 cells incubated in SFM for 24 hr.

FIG. 23B is a plot showing the relative migration distance (microns) for 10 cells incubated in PDGF-BB [1.4 nM] for 24 hr.

FIG. 23C is a dot plot showing on the abscissa the 24-hr integrated cytoplasmic FoxO1-clover localization and on the ordinate the 24-hr migration distance of individual cells incubated in SFM (red dots) or PDGF-BB ([1.4 nM], blue dots).

FIG. 23D is a dot plot of the 24-hr migration distance of individual cells (n=200) treated with SFM (purple), EGF ([4.2 nM], red), PDGF-AA ([1.4 pM], orange), PDGF-BB ([1.4 pM], green), or IGF-I ([500 pM], blue). Cells have been separated into those that exited G1 during the tracking period (+, left side) from those remaining in G1 (−, right side), based on redistribution of the HDHB-mKate2 reporter molecule.

FIG. 24A is a dot plot showing on the ordinate the 24-hr integrated Akt signaling activity (cytoplasmic FoxO1-clover localization) and on the abscissa the 24-hr migration distance of individual cells incubated in SFM (purple) or IGF-I [500 pM]±different concentrations of linsitinib, as indicated.

FIG. 24B is a plot showing the integrated Akt signaling activity over time for cells incubated with IGF-I [500 pM] for 24 hr. The black circles represents the time an individual cell entered S-phase.

FIG. 25A is a dot plot of the 24-hr migration distance of individual cells (n=200) treated with SFM (purple), IGF-I ([500 pM], blue), and IGF-I plus linsitinib ([50 nM], green), [100 nM], orange), or [200 nM], red). Cells have been separated into those that exited G1 during the tracking period (+, left side) from those remaining in G1 (−, right side), based on redistribution of the HDHB-mKate2 reporter molecule.

FIG. 25B is a dot plot of the data in 25A with the integrated 24-hour Akt signaling data on the ordinate and the 24 hour migration on the abscissa.

FIG. 25C is a plot showing the integrated 24-hour migration distance over time for cells incubated with IGF-1 [500 pM] for 24 hours The black circles represents the time an individual cell entered S-phase.

### SEQUENCE LISTING

SEQ ID NO: 1 is a sequence of a mutated form of mouse FOXO1.

SEQ ID NO: 2 is a sequence of a mutated form of human FOXO1.

SEQ ID NO: 3 is a sequence derived from human DNA helicase b

SEQ ID NO: 4 is a sequence of gfpCLOVER.

SEQ ID NO: 5 is a sequence of mKate.

### DETAILED DESCRIPTION

Described herein is a sensor that measures Akt activity and the application of a set of tools for measuring signaling in individual cells within a population. The reporter protein is based on FOXO1, an Akt substrate that transits between the nucleus and cytoplasm (Brunet A et al, Cell 96, 857-868 (1999); Rena G et al, J Biol Chem 274, 17179-17183 (1999); Rena G et al, EMBO J 21, 2263-2271 (2002); Van Der Heide L P et al, Biochem J 380, 297-309 (2004); Woods Y L et al, Biochem J 355, 597-607 (2001); Zhang X L et al, J Biol Chem 277, 45276-45284 (2002); all of which are incorporated by reference herein). The subcellular movement of the reporter is readily tracked in living cells through its fusion to Clover, a highly fluorescent modified EGFP (Lam A J et al, Nat Methods 9, 1005-1012 (2012); incorporated by reference herein). This reagent enables quantification of the rate and extent of changes in Akt activity over time, and shows by analyzing single cells that Akt signaling is highly heterogeneous in response to the same stimulus. The methods described herein can better map the Akt pathway functions under a range of biological conditions in different cell types.

### Terms

Unless otherwise noted, technical terms are used according to conventional usage. Definitions of common terms in molecular biology may be found in Benjamin Lewin, Genes V, published by Oxford University Press, 1994 (ISBN 0-19-854287-9);

Kendrew et al. (eds.), The Encyclopedia of Molecular Biology, published by Blackwell Science Ltd., 1994 (ISBN 0-632-02182-9); and Robert A. Meyers (ed.), Molecular
Biology and Biotechnology: a Comprehensive Desk Reference, published by VCR Publishers, Inc., 1995 (ISBN 1-56081-569-8).

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The term “comprises” means “includes.”

In addition, the materials, methods, and examples are illustrative only and not intended to be limiting. In order to facilitate review of the various embodiments of the disclosure, the following explanations of specific terms are provided:

Agonist: An agonist is an agent, such as a small molecule or protein that binds to a protein and activates the protein to produce a particular biological response. An agonist can be a naturally occurring or artificially synthesized compound. For example, an Akt1 agonist is an agent that activates and/or increases the activity of Akt1.

Antagonist: An antagonist is an agent, such as a small molecule or protein that binds to a protein and prevents or stops the protein from producing a particular biological response. An antagonist can be a naturally occurring or artificially synthesized compound. For example, an Akt1 agonist is an agent that activates and/or increases the activity of Akt1. An antagonist can also be called an inhibitor and the terms can be used interchangeably.

Antibody: A polypeptide including at least a light chain or heavy chain immunoglobulin variable region which specifically recognizes and binds an epitope of an antigen, such as a form of FOXO1 described herein as SEQ ID NO: 1, SEQ ID NO: 2 or other homolog thereof or a protein tag covalently or otherwise complexed thereto. Antibodies are composed of a heavy and a light chain, each of which has a variable region, termed the variable heavy (VH) region and the variable light (VL) region. Together, the VH region and the VL region are responsible for binding the antigen recognized by the antibody.

The term “antibody” encompasses intact immunoglobulins, as well the variants and portions thereof, such as Fab fragments, Fab′ fragments, F(ab)′2 fragments, single chain Fv proteins (“scFv”), and disulfide stabilized Fv proteins (“dsFv”). A scFv protein is a fusion protein in which a light chain variable region of an immunoglobulin and a heavy chain variable region of an immunoglobulin are bound by a linker. In dsFvs the chains have been mutated to introduce a disulfide bond to stabilize the association of the chains. The term also includes genetically engineered forms such as chimeric antibodies, heteroconjugate antibodies (such as, bispecific antibodies). See also, Pierce Catalog and Handbook, 1994-1995 (Pierce Chemical Co., Rockford, Ill.); Kuby, J., Immunology, 3rd Ed., W.H. Freeman & Co., New York, 1997.

Binding or stable binding: An association between two substances or molecules, such as the association of an antibody with a peptide, nucleic acid to another nucleic acid, or the association of a protein with another protein or nucleic acid molecule, or the association of a small molecule drug with a protein or other biological macromolecule. Binding can be detected by any procedure known to one skilled in the art, such as by physical or functional properties. For example, binding can be detected functionally by determining whether binding has an observable effect upon a biosynthetic process such as expression of a gene, DNA replication, transcription, translation, protein activity and the like.

Conservative variants: A substitution of an amino acid residue for another amino acid residue having similar biochemical properties. “Conservative” amino acid substitutions are those substitutions that do not substantially affect or decrease an activity of an MHC Class II polypeptide, such as an MHC class II al polypeptide. A polypeptide can include one or more amino acid substitutions, for example 1-10 conservative substitutions, 2-5 conservative substitutions, 4-9 conservative substitutions, such as 1, 2, 5 or 10 conservative substitutions. Specific, non-limiting examples of a conservative substitution include the following examples:

| Original Amino Acid | Conservative Substitutions |
|---|---|
| Ala | Ser |
| Arg | Lys |
| Asn | Gln, His |
| Asp | Glu |
| Cys | Ser |
| Gln | Asn |
| Glu | Asp |
| His | Asn; Gln |
| Ile | Leu, Val |
| Leu | Ile; Val |
| Lys | Arg; Gln; Glu |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

Contacting: Placement in direct physical association, including contacting of a solid with a solid, a liquid with a liquid, a liquid with a solid, or either a liquid or a solid with a cell or tissue, whether in vitro or in vivo. Contacting can occur in vitro with isolated cells or tissue or in vivo by administering to a subject.

Fluorescent protein: A protein characterized by a barrel structure that allows the protein to absorb light and emit it at a particular wavelength. Fluorescent proteins include green fluorescent protein (GFP) modified GFPs and GFP derivatives (such as Clover) and other fluorescent proteins, such as EGFP, EBFP, YFP, BFP, CFP, ECFP, and circularly permutated fluorescent proteins such as cpVenus.

Label: A label may be any substance capable of aiding a machine, detector, sensor, device, column, or enhanced or unenhanced human eye from differentiating a labeled composition from an unlabeled composition. Labels may be used for any of a number of purposes and one skilled in the art will understand how to match the proper label with the proper purpose. Examples of uses of labels include purification of biomolecules, identification of biomolecules, detection of the presence of biomolecules, detection of protein folding, and localization of biomolecules within a cell, tissue, or organism. Examples of labels include but are not limited to: radioactive isotopes (such as carbon-14 or ¹⁴C) or chelates thereof; dyes (fluorescent or nonfluorescent), stains, enzymes, nonradioactive metals, magnets, protein tags, any antibody epitope, any specific example of any of these; any combination between any of these, or any label now known or yet to be disclosed. A label may be covalently attached to a biomolecule or bound through hydrogen bonding, Van Der Waals or other forces. A label may be covalently or otherwise bound to the N-terminus, the C-terminus or any amino acid of a polypeptide or the 5′ end, the 3′ end or any nucleic acid residue in the case of a polynucleotide.

One particular example of a label is a protein tag. A protein tag comprises a sequence of one or more amino acids that may be used as a label as discussed above, particularly for use in protein purification. In some examples, the protein tag is covalently bound to the polypeptide. It may be covalently bound to the N-terminal amino acid of a polypeptide, the C-terminal amino acid of a polypeptide or any other amino acid of the polypeptide. Often, the protein tag is encoded by a polynucleotide sequence that is immediately 5′ of a nucleic acid sequence coding for the polypeptide such that the protein tag is in the same reading frame as the nucleic acid sequence encoding the polypeptide. Protein tags may be used for all of the same purposes as labels listed above and are well known in the art. Examples of protein tags include chitin binding protein (CBP), maltose binding protein (MBP), glutathione-S-transferase (GST), poly-histidine (His), thioredoxin (TRX), FLAG®, V5, c-Myc, HA-tag, fluorescent proteins, and so forth.

A His-tag facilitates purification and binding to on metal matrices, including nickel matrices, including nickel matrices bound to solid substrates such as agarose plates or beads, glass plates or beads, or polystyrene or other plastic plates or beads. Other protein tags include BCCP, calmodulin, Nus, Thioredoxin, Streptavidin, SBP, and Ty, or any other combination of one or more amino acids that can work as a label described above.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Operably linked DNA sequences can be contiguous and, where necessary to join two protein-coding regions, in the same reading frame. In some examples, a promoter sequence is operably linked to a protein coding sequence, such that the promoter drives transcription of the linked nucleic acid and/or expression of the protein.

Promoter: Promoters are sequences of DNA near the 5′ end of a gene that act as a binding site for RNA polymerase, and from which transcription is initiated. A promoter includes necessary nucleic acid sequences near the start site of transcription, such as, in the case of a polymerase II type promoter, a TATA element. A promoter can include an enhancer. A promoter can also include a repressor element.

Promoters can be constitutively active, such as a promoter that is continuously active and is not subject to regulation by external signals or molecules. In some examples, a constitutive promoter is active such that expression of a sequence operably linked to the promoter is expressed ubiquitously (for example, in all cells of a tissue or in all cells of an organism and/or at all times in a single cell or organism, without regard to temporal or developmental stage).

An inducible promoter is a promoter that has activity that is increased (or that is de-repressed) by some change in the environment of the cell such as the addition of a particular agent to the cell media or a removal of a nutrient or other component from the media of the cell.

Polypeptide: A polymer in which the monomers are amino acid residues which are joined together through amide bonds. When the amino acids are alpha-amino acids, either the L-optical isomer or the D-optical isomer can be used, the L-isomers being preferred. The terms “polypeptide” or “protein” or “peptide” as used herein are intended to encompass any amino acid sequence and include modified sequences such as glycoproteins. The term “polypeptide” or “protein” or “peptide” is specifically intended to cover naturally occurring proteins, as well as those which are recombinantly or synthetically produced. It should be noted that the term “polypeptide” or “protein” includes naturally occurring modified forms of the proteins, such as glycosylated, phosphorylated, or ubiquinated forms.

Recombinant: A recombinant nucleic acid or polypeptide is one that has a sequence that is not naturally occurring or has a sequence that is made by an artificial combination of two or more otherwise separated segments of sequence (such as a FOXO1 homolog in combination with a fluorescent protein). This artificial combination is often accomplished by chemical synthesis or, more commonly, by the artificial manipulation of isolated segments of nucleic acids, e.g., by genetic engineering techniques. A recombinant polypeptide can also refer to a polypeptide that has been made using recombinant nucleic acids, including recombinant nucleic acids transferred to a host organism that is not the natural source of the polypeptide.

Sequence identity/similarity: The identity/similarity between two or more nucleic acid sequences, or two or more amino acid sequences, is expressed in terms of the identity or similarity between the sequences. Sequence identity can be measured in terms of percentage identity; the higher the percentage, the more identical the sequences are. Sequence similarity can be measured in terms of percentage similarity (which takes into account conservative amino acid substitutions); the higher the percentage, the more similar the sequences are.

Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations.

The NCBI Basic Local Alignment Search Tool (BLAST) (Altschul et al., J. Mol. Biol. 215:403-10, 1990) is available from several sources, including the National Center for Biological Information (NCBI, National Library of Medicine, Building 38A, Room 8N805, Bethesda, Md. 20894) and on the Internet, for use in connection with the sequence analysis programs blastp, blastn, blastx, tblastn and tblastx. Additional information can be found at the NCBI web site. BLASTN is used to compare nucleic acid sequences, while BLASTP is used to compare amino acid sequences. If the two compared sequences share homology, then the designated output file will present those regions of homology as aligned sequences. If the two compared sequences do not share homology, then the designated output file will not present aligned sequences.

Once aligned, the number of matches is determined by counting the number of positions where an identical nucleotide or amino acid residue is presented in both sequences. The percent sequence identity is determined by dividing the number of matches either by the length of the sequence set forth in the identified sequence, or by an articulated length (such as 100 consecutive nucleotides or amino acid residues from a sequence set forth in an identified sequence), followed by multiplying the resulting value by 100. For example, a nucleic acid sequence that has 1166 matches when aligned with a test sequence having 1154 nucleotides is 75.0 percent identical to the test sequence (1166÷1554*100=75.0). The percent sequence identity value is rounded to the nearest tenth. For example, 75.11, 75.12, 75.13, and 75.14 are rounded down to 75.1, while 75.15, 75.16, 75.17, 75.18, and 75.19 are rounded up to 75.2. The length value will always be an integer. In another example, a target sequence containing a 20-nucleotide region that aligns with 20 consecutive nucleotides from an identified sequence as follows contains a region that shares 75 percent sequence identity to that identified sequence (that is, 15÷20*100=75).

For comparisons of amino 5 acid sequences of greater than about 30 amino acids, the Blast 2 sequences function is employed using the default BLOSUM62 matrix set to default parameters, (gap existence cost of 11, and a per residue gap cost 5 of 1). Homologs are typically characterized by possession of at least 70% sequence identity counted over the full-length alignment with an amino acid sequence using the NCBI Basic Blast 2.0, gapped blastp with databases such as the nr or swissprot database. Queries searched with the blastn program are filtered with DUST (Hancock and Armstrong, 1994, Comput. Appl. Biosci. 10:67-70). Other programs use SEG. In addition, a manual alignment can be performed. Proteins with even greater similarity will show increasing percentage identities when assessed by this method, such as at least about 75%, 80%, 85%, 90%, 95%, 98%, or 99% sequence identity to a protein.

When aligning short peptides (fewer than around 30 amino acids), the alignment is performed using the Blast 2 sequences function, employing the PAM30 matrix set to default parameters (open gap 9, extension gap 1 penalties). Proteins with even greater similarity to the reference sequence will show increasing percentage identities when assessed by this method, such as at least about 60%, 70%, 75%, 80%, 85%, 90%, 95%, 98%, or 99% sequence identity to a protein. When less than the entire sequence is being compared for sequence identity, homologs will typically possess at least 75% sequence identity over short windows of 10-20 amino acids, and can possess sequence identities of at least 85%, 90%, 95% or 98% depending on their identity to the reference sequence. Methods for determining sequence identity over such short windows are described at the NCBI web site.

One of skill in the art will appreciate that the particular sequence identity ranges are provided for guidance only. A pair of proteins or nucleic acids with 50%, 60%, 70%, 75%, 80%, 85%, 90%, 95%, 98%, or 99% identity to one another can be termed ‘homologs,’ particularly if they perform the same function as one another, even more particularly if they perform the same function to substantially the same degree, and still more particularly if they perform the same function substantially equivalently. One of skill in the art in light of this disclosure, particularly in light of the Examples below, would be able to determine without undue experimentation whether or not a given protein or nucleic acid sequence with 50%, 60%, 70%, 75%, 80%, 85%, 90%, 95%, 98%, or 99% identity to the sequences listed herein is a homolog to the sequences listed herein. Homologs need not be the same length as the biological molecules listed herein and may include truncations (fewer amino acids or nucleotides) or extensions (more amino acids or nucleotides) relative to the biological molecules listed herein. In one example, SEQ ID NO: 1 and SEQ ID NO: 2 are homologs of one another.

### Methods of Selecting Test Compounds

Disclosed herein are methods of selecting test compounds that affect Akt activity. Such methods involve adding a test compound to a cell that expresses both Akt and a FOXO1 biosensor, the FOXO1 biosensor comprising mutations at S212, H215, and S218 of the human homolog of FOXO1, or at the equivalent positions in other homologs (such as the mouse homolog of FOXO1). A mouse homolog comprising exemplary mutations is described as SEQ ID NO: 1 herein. A human homolog comprising exemplary mutations is described as SEQ ID NO: 2 herein. In some examples, the cell expresses the FOXO1 biosensor because it was previously transfected with an expression vector that has a nucleic acid sequence that expresses the FOXO1 biosensor operably linked to a promoter that drives expression of FOXO1 biosensor. The cell can express Akt endogenously or it can express Akt because it was previously transfected with an expression vector comprising Akt or a mutant thereof (such as a constitutively active mutant thereof). The biosensor can be labeled with any label described herein or known in the art. In some examples, the label comprises a fluorescent protein.

A test compound is generally provided in a vehicle, such as a solvent. The vehicle can be any appropriate solvent including compositions comprising water, ions, or organic compounds. Examples of vehicles include buffered saline or other buffered solvents or DMSO or other organic solvents.

When Akt is activated, it in turn phosphorylates the described FOXO1 biosensor. When the FOXO1 biosensor is unphosphorylated it accumulates the nucleus. When it is phosphorylated, it is excluded from the nucleus and accumulates in the cytoplasm. Therefore, when a cell expressing the biosensor is provided in a serum free media that lacks any compounds that activate Akt (such as IGF-1, fetal bovine serum or PDGF-ββ) then the biosensor will accumulate in the nucleus. A test compound that acts as an Akt agonist will activate Akt which will in turn phosphorylate the biosensor and cause the biosensor to exit the nucleus. A decrease in the relative nuclear intensity of the biosensor over time in a cell under the above conditions contacted with the test compound is an indication that the test compound is an agonist of Akt. In particular, it is an indication when the decrease is greater than that of a negative control cell expressing the biosensor that was contacted with the vehicle and not the test compound.

A cell expressing the biosensor provided in a media that includes one or more compounds that activate Akt will have much of the FOXO1 biosensor excluded from the nucleus and present in the cytoplasm. A test compound that acts as an Akt antagonist will inhibit Akt, reducing the amount of phosphorylation of the biosensor and causing the biosensor to traffic to the nucleus. Therefore an increase in the relative nuclear intensity of the biosensor over time in a cell contacted with the test compound under the above conditions is an indication that the test compound is an antagonist of Akt. In particular, it is an indication when the increase is greater than that of a negative control cell expressing the biosensor that was contacted with the vehicle and not the test compound.

The FOXO1 biosensor can be located by any method known in the art. It can be located by an antibody specific for the FOXO1 biosensor. The antibody specific for the FOXO1 biosensor can be labeled itself (for example with a fluorescent molecule) or the antibody can be detected with a second labeled antibody specific for the antibody that is specific for the FOXO1 biosensor. In one example of such a system, the FOXO1 biosensor can be detected with an unlabeled rabbit anti-FOXO1 antibody and the rabbit antibody can be detected with a labeled mouse anti-rabbit antibody.

In other examples, the biosensor further comprises a protein tag. The protein tag is expressed in-frame with FOXO1 biosensor. In further examples, the protein tag is expressed at the N- or C-terminus of the FOXO1 biosensor. The protein tag can be any tag that aids in the detection of the FOXO1 biosensor such as an antibody epitope, avidin or streptavidin (both of which can be detected by labeled biotin), or a fluorescent protein. One example of such a fluorescent protein is Clover fluorescent protein (SEQ ID NO: 4.)

A test compound can be any compound that is suspected of effecting Akt activity. Examples of test compounds include small molecules, proteins, peptides, or other potential therapeutic compounds. A test compound can also be a compound known to inhibit Akt activity that is used as a positive control. A test compound can also be a compound known not to affect Akt activity that is used as a negative control. The methods herein can be used to screen a plurality of test compounds, also described as a library of test compounds. The methods herein can be further adapted to high throughput screening of a set of test compounds in batches of 96, 384, or 1048 on assay plates adapted for such screening.

In still further examples, different concentrations of the test compound can be contacted with the cell, thereby creating a dose response curve. More specific examples are described below.

### EXAMPLES

The following examples are illustrative of disclosed methods. In light of this disclosure, those of skill in the art will recognize that variations of these examples and other examples of the disclosed method would be possible without undue experimentation.

### Example 1

### Developing a Reporter to Track Akt Activity in Living Cells

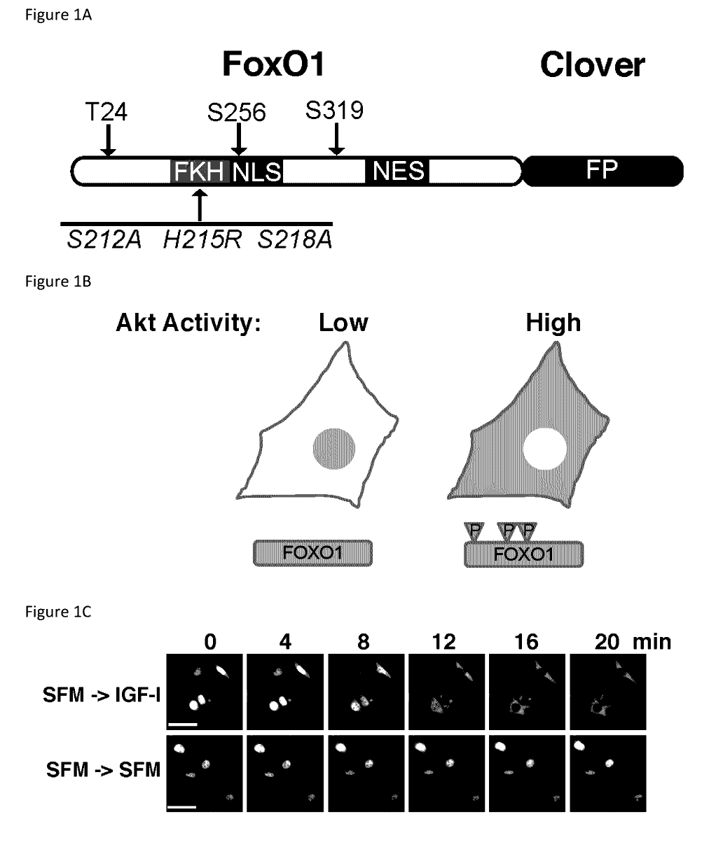

Disclosed herein is a fluorescent fusion protein used to assess Akt activity at the single cell level. The fusion protein is based on FOXO1, a well-characterized Akt kinase substrate (Hay, 2011 supra). FOXO1 contains three Akt phosphorylation sites that modulate the functions of nuclear localization (NLS) and nuclear export (NES) motifs (FIG. 1A). NLS activity is inhibited by Akt phosphorylation, and NES activity is enhanced, shifting the equilibrium of subcellular localization from the nucleus to the cytoplasm (Brunet et al, 1999 supra; Rena et al, 1999 supra; Rena et al, 2002 supra; Zhang et al, 2002 supra) (FIG. 1B). The reporter was constructed by fusing the green fluorescent protein, Clover (Lam et al, 2012 supra), to the COOH-terminus of FOXO1. Additionally, an engineered three amino acid substitution was made in the Forkhead domain of FOXO1 to inhibit its DNA binding activity (Tang E D et at J Biol Chem 274, 16741-16746 (1999); incorporated by reference herein), thus rendering the construct transcriptionally inactive, and preventing effects of phosphorylation by the protein kinase, Mst1 (Lehtinen M K et al, Cell 125, 987-1001 (2006); incorporated by reference herein) (FIG. 1A). After lentiviral delivery into mouse 10T1/2 fibroblasts, stable selection, and cell sorting, rapid and robust reporter transit from the nucleus to the cytoplasm in response to the growth factor, IGF-I (FIG. 1B, FIG. 1C) were demonstrated.

### Example 2

### Dynamic Localization of the FOXO1-Clover Reporter Protein in Cycling Cells

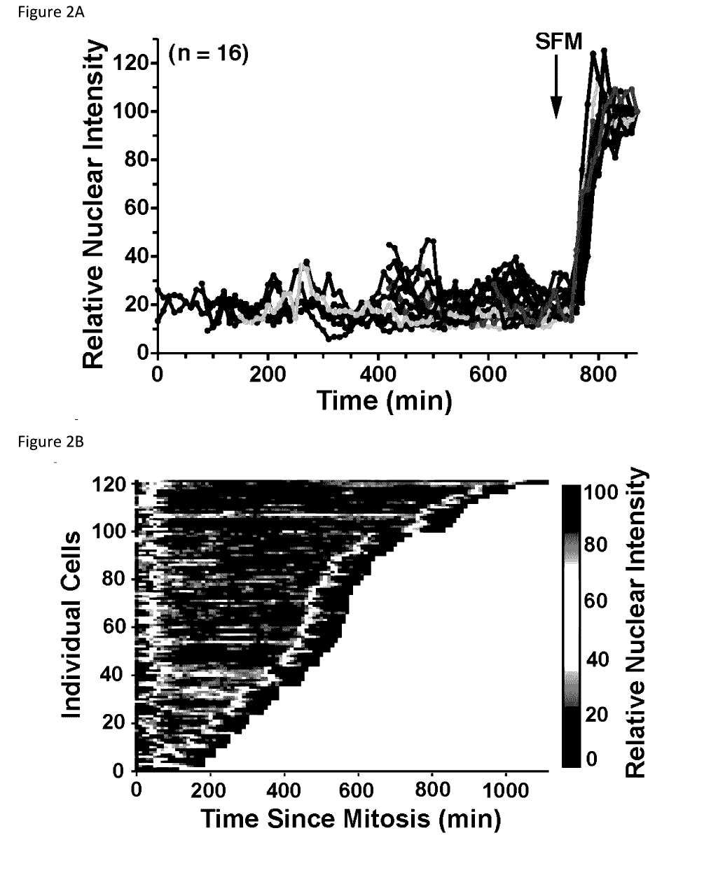

To test the behavior of the reporter protein over time, 10T1/2 fibroblasts were tracked during a 12-hr incubation in medium with 10% FBS. The medium was then replaced with serum-free medium, and cells were imaged for a further 90 min. It was found that in the presence of 10% FBS the localization of the reporter in the cytoplasm was stable, exhibiting only minor oscillations (4% average absolute deviation from the mean). Moreover, removal of serum caused a rapid rise in nuclear fluorescence that was maintained for the 90-min incubation period (FIG. 2A). During long-term incubation in serum-containing medium many of the cells underwent at least one division. When individual fibroblasts were aligned based on their time since the last mitosis, the fractional localization of the reporter in the nucleus was relatively constant, was independent of the time since cell division, and ranged from 10-30% of that measured in serum-free medium (FIG. 2B). Taken together, the results in FIGS. 2A and 2B show that exposure to 10% FBS caused sustained Akt kinase activity that maintained the reporter protein primarily in the cytoplasm, and that Akt kinase activity did not vary significantly during the cell cycle.

It is likely that structural factors such as changes in nuclear shape or volume can influence the apparent nuclear accumulation of the FOXO1-clover reporter protein. These alterations as well as technical issues can contribute to measurement errors in the described cell tracking process. To assess potential measurement errors, tracked images of 5 cells were re-analyzed up to 10-times during a 60-minute incubation in serum-free medium. Under these experimental conditions, we found that the intensity of nuclear fluorescence varied on average by only 3% from the mean value, although some cells exhibited greater variability than others. As this value is smaller than the mean variability observed in cells incubated in serum-containing medium (FIG. 2A), the results suggest that the disclosed experimental system provides a sensitive readout of biological factors that act on the subcellular location of FOXO1, and that it is not influenced significantly by changes in cell shape or volume.

### Example 3

### Assessing Growth Factor Specificity and Responsiveness of the FOXO1-Clover Reporter to Akt-Mediated Signaling

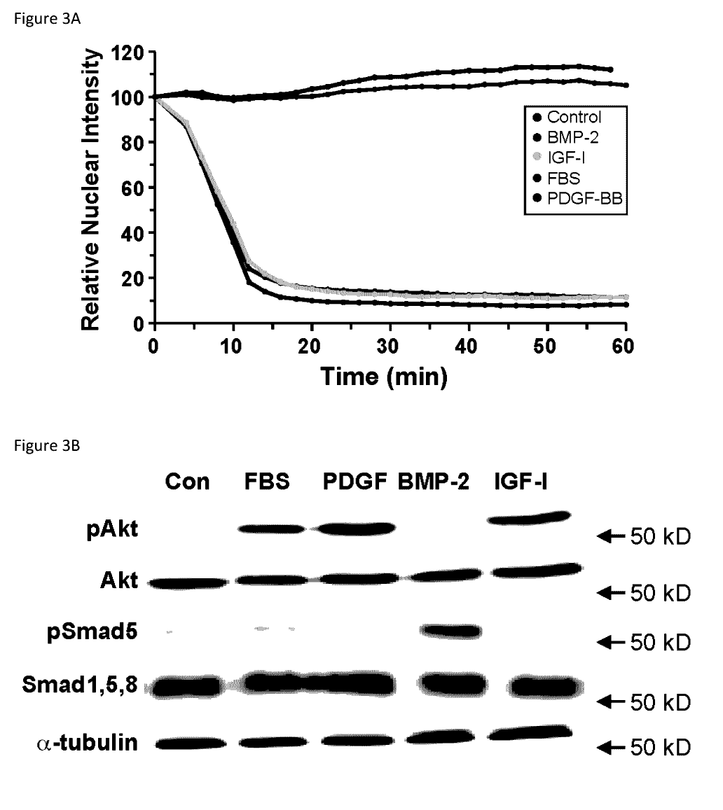

Serum-starved 10T1/2 cells were treated with 10% FBS or with individual growth factors in serum-free medium, and the subcellular localization of FOXO1-clover was tracked for 60 minutes. Cells incubated with FBS, PDGF-BB, or R3-IGF-I showed rapid and sustained translocation of the reporter from the nucleus to the cytoplasm in parallel with stimulation of Akt phosphorylation (FIGS. 3A and 3B). In contrast, cells maintained in serum-free medium or treated with BMP-2 had predominantly nuclear localization of FOXO1-clover, and exhibited minimal Akt phosphorylation. BMP-2 did stimulate phosphorylation of Smad5, one of its key intracellular signaling proteins (Katagiri T and Tsukamoto S, Biol Chem 394, 703-714 (2013); Wang R N et al, Genes Dis 1, 87-105 (2014); both of which are incorporated by reference herein), indicating that its addition did cause cognate receptor activation in 10T1/2 cells (FIGS. 3A and 3B).

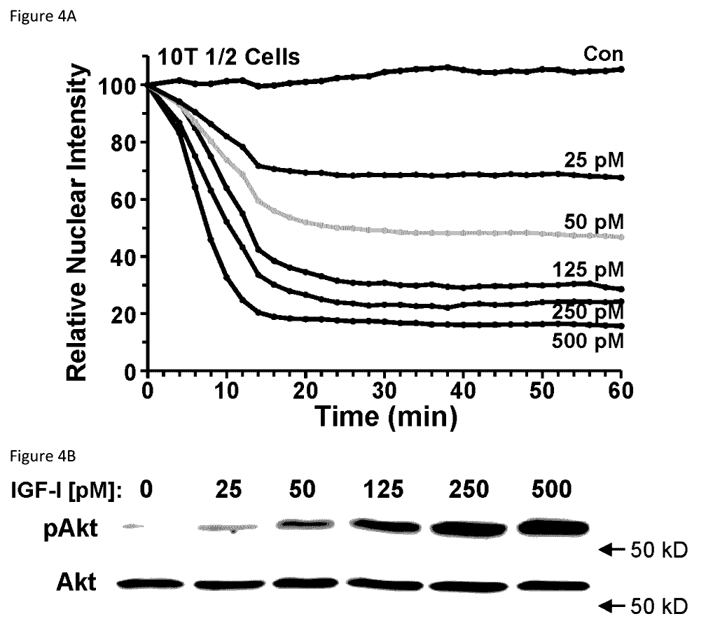

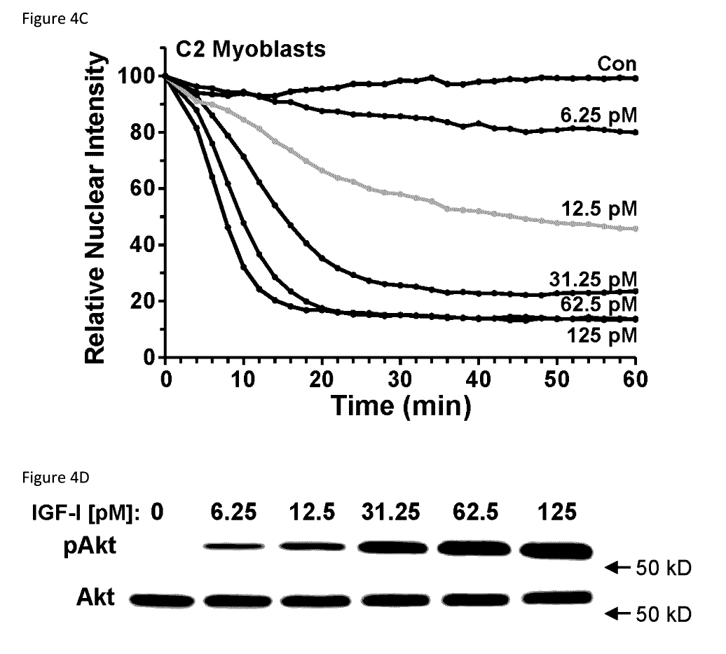

The effect of different concentrations of R3-IGF-I on the rate and extent of cytoplasmic accumulation of the FOXO1-clover reporter protein was assessed. In serum-free medium, the reporter was predominantly in the nucleus of 10T1/2 cells (FIG. 4A). Addition of R3-IGF-I caused a rapid and dose-dependent reduction in nuclear levels of the reporter, with half-maximal translocation being reached by 8-10 min after onset of incubation, and maximal values being attained within 14-16 min (FIG. 4A). Similar results were seen in C2 myoblasts, but with a marked increase in sensitivity to IGF-I (FIG. 4C, compare with FIG. 4A), and yet a slower rate of cytoplasmic accumulation at the two lowest growth factor concentrations (FIG. 4C). Since R3-IGF-I binds minimally to IGF binding proteins, which typically inhibit acute IGF actions (Bach L A et al, Trends Endocrinol Metab 16, 228-234 (2005); Baxter R C, Nat Rev Cancer 14, 329-341 (2014); incorporated by reference herein), IGF binding proteins are probably not responsible for the variable responsiveness seen between these two cell types.

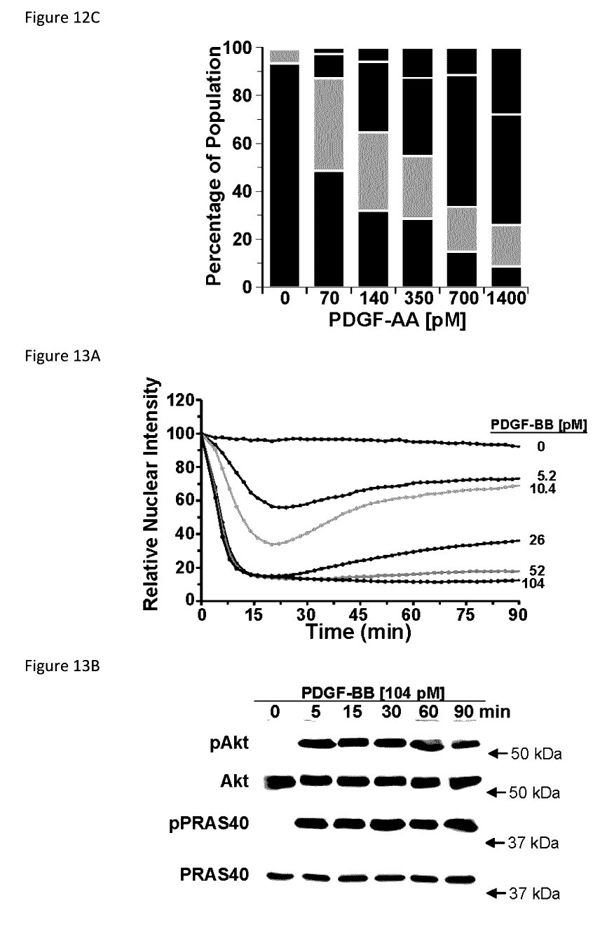

To confirm that reporter localization was tracking Akt activity, Akt phosphorylation was measured by immunoblotting whole cell protein lysates from the same cells studied FIGS. 12A and C. In both 10T1/2 cells and C2 myoblasts, the addition of IGF-I caused a dose-dependent increase in the extent of Akt phosphorylation (FIGS. 4B and 4D). Thus, there is a direct correspondence between the cytoplasmic localization of the FOXO1-clover reporter and the amount of Akt phosphorylation in response to treatment with IGF-I.

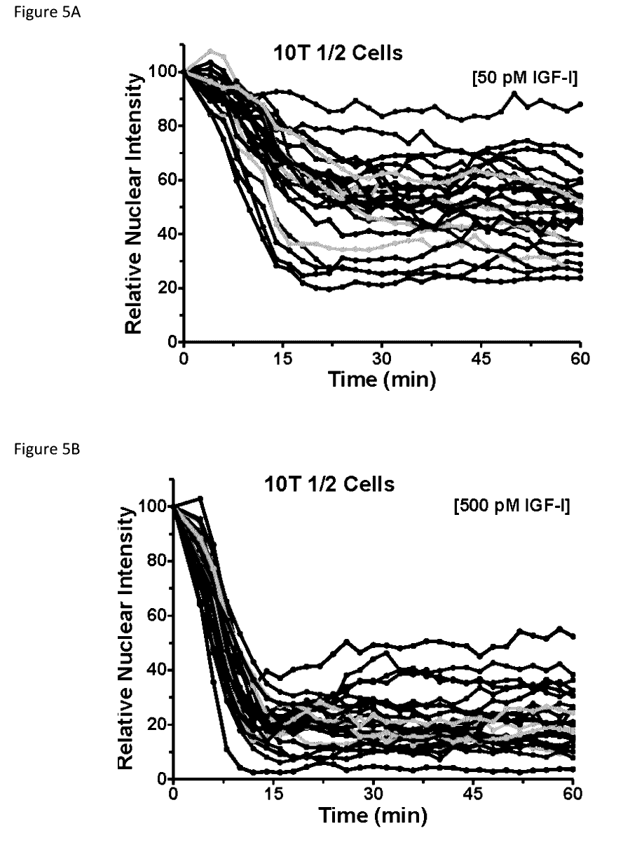

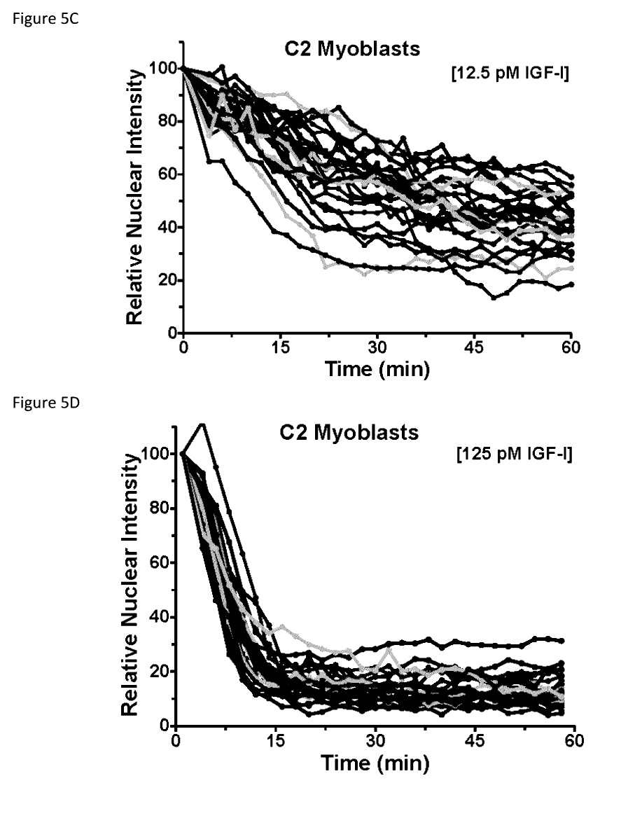

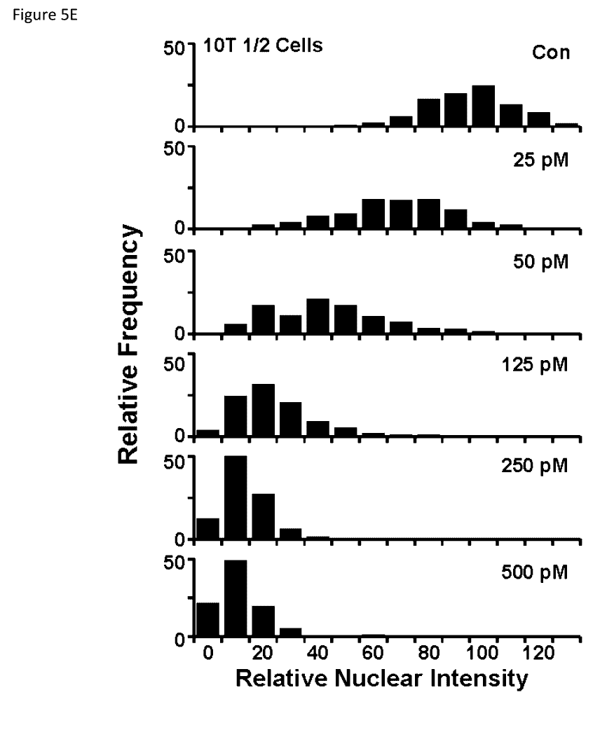

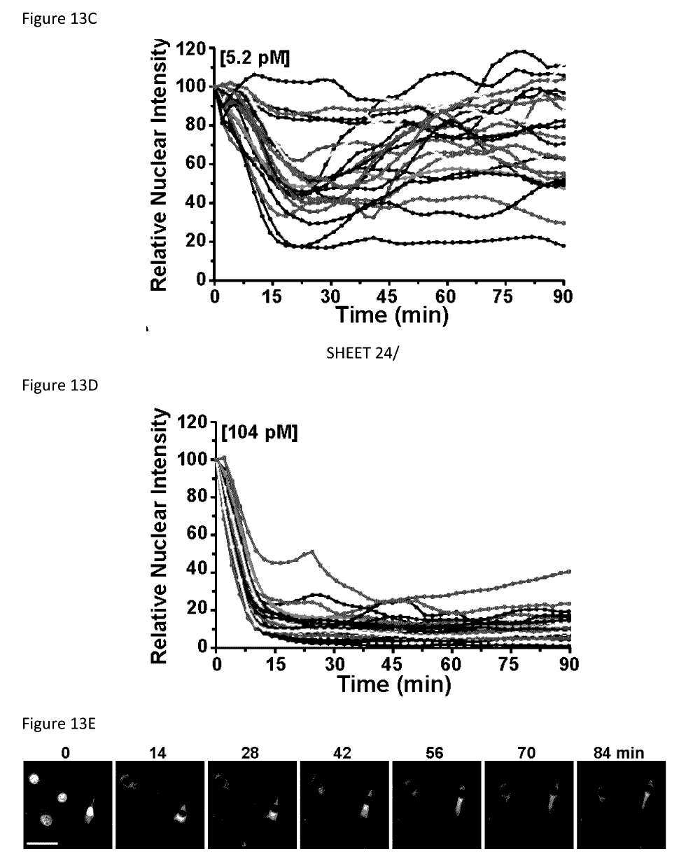

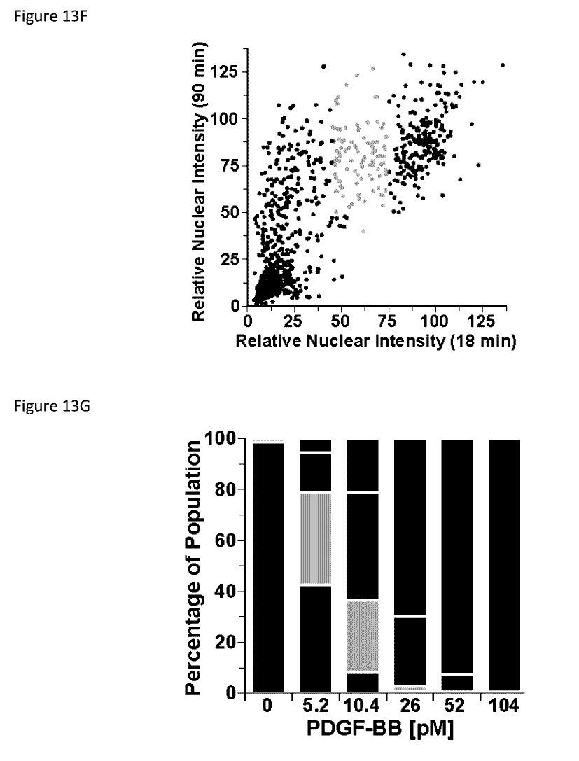

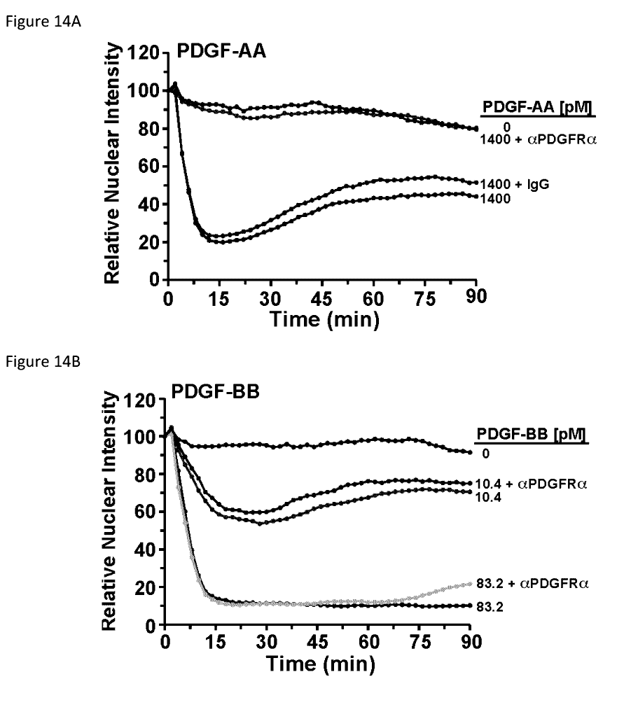

The time-course studies and immunoblotting results in FIGS. 4A-4D represent population averages, and thus do not provide insight into the behavior of individual cells exposed to different concentrations of IGF-I. Single cell responses were then separated from population results. It was found that responsiveness to IGF-I was markedly variable at lower growth factor concentrations for both 10T1/2 cells [50 pM] and C2 myoblasts [12.5 pM] (FIG. 13A, 13C). However, at higher levels of growth factor exposure ([500 pM] for 10T1/2 cells, [125 pM] for C2 cells), initial rates of export of FOXO1-clover from the nucleus were more uniform that at low IGF-I concentrations, but there was still substantial heterogeneity in the amount of reporter remaining in the cytoplasm (FIGS. 5B and 5D). A more in depth examination of these observations is depicted in FIGS. 5E and F, which illustrate by frequency plots the range of signaling responses in both 10T1/2 and C2 cells during incubation with different IGF-I concentrations for 60 min. We interpret these results to indicate that the effects of a given dose of IGF-I on signaling in individual cells may be quite variable, even within populations that appear to respond in consistent ways.

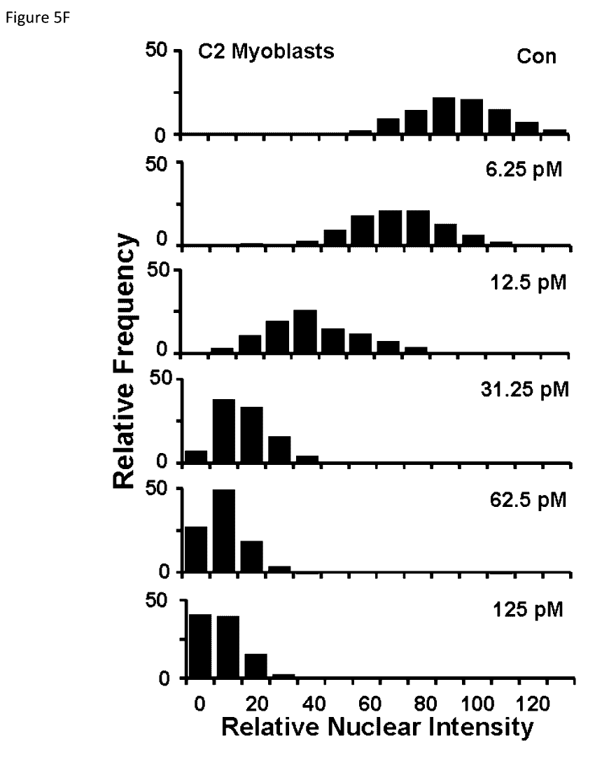

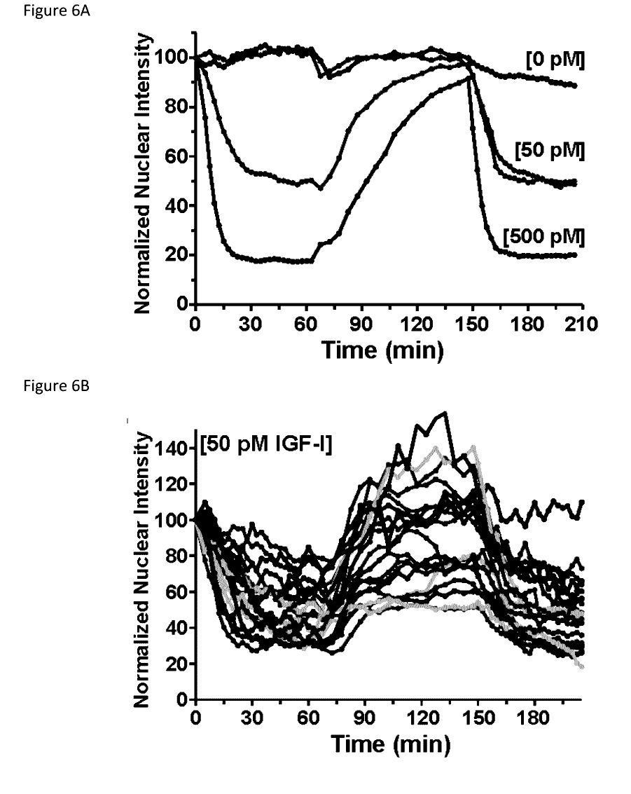

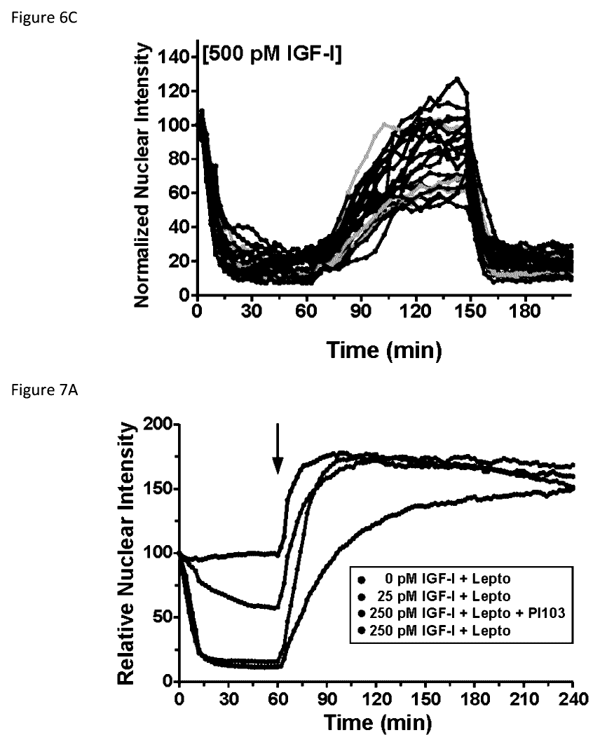

The effects of repeated exposures to IGF-I on the behavior of the FOXO1-clover reporter were also tested. Cells were incubated with R3-IGF-I for 60 min, followed by a 90-min washout period in serum-free medium, and then by a second incubation in IGF-I-containing medium. We found that 10T1/2 cells treated sequentially with IGF-I exhibited qualitatively similar population responses to each dose (FIG. 6A). Moreover, the second response to IGF-I [50 pM] closely matched results in cells treated only during the second time period (FIG. 6A, compare red and green tracings). When results from 25 single cells from the population presented in FIG. 6A were examined, there was significant individual variability at both growth factor doses compared with the population mean (FIGS. 6B and 6C). This was particularly evident in cells incubated with the lower IGF-I concentration [50 pM], in which there was little correspondence between responses to the first and second treatments (FIG. 6B). Thus, as observed in FIGS. 5A-5F, live-cell imaging reveals marked variability in individual cellular responses to IGF-I, illustrating a complexity in signaling behaviors that is masked when only population data are analyzed.

### Example 4

### Altering the Sub-Cellular Equilibrium of the FOXO1-Clover Reporter by Blocking Nuclear Export

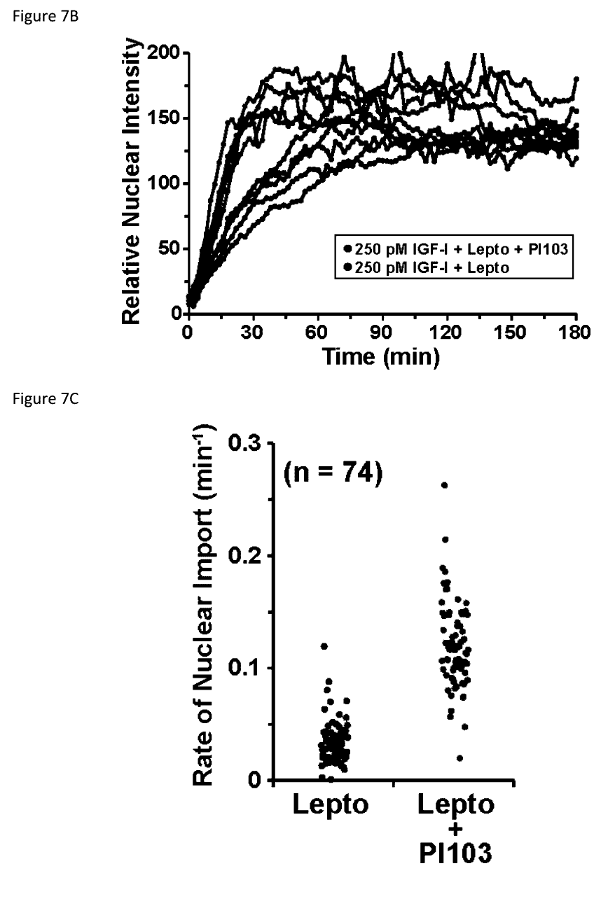

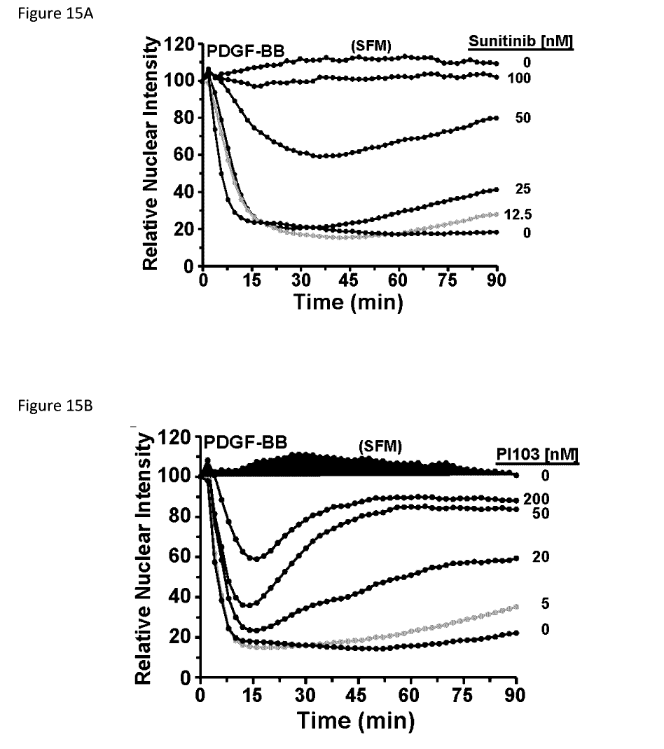

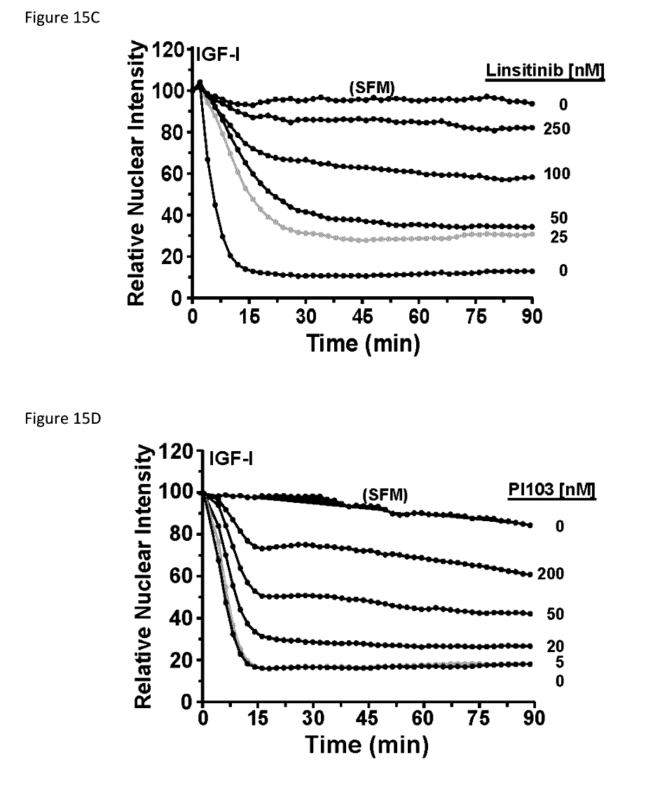

The kinetics of sub-cellular translocation of the reporter protein was then assessed. Cells were incubated with different concentrations of IGF-I for 60 min to promote movement of FOXO1-clover into the cytoplasm, followed by addition of leptomycin B, an inhibitor of nuclear export (Wolff B et al, Chem Biol 4, 139-147 (1997); incorporated by reference herein). Exposure of cells to leptomycin B led to re-accumulation of the reporter in the nucleus, but at a rate that was inversely related to the prior dose of IGF-I. At the higher growth factor concentration [250 pM], the half-maximal time of nuclear appearance after leptomycin B was ^{˜}25 min, while in the presence of the lower dose [25 pM], it was ^{˜}5 min (FIG. 7A). Furthermore, acutely reducing PI3-kinase activity (and thus Akt) with the dual-purpose inhibitor, PI103 (which also blocks mTorc2 (Fan Q W et al, Cancer Cell 9, 341-349 (2006); incorporated by reference herein), enhanced the effect of leptomycin, and led to an accelerated rate of nuclear accumulation of the FOXO1-clover reporter protein, even in cells treated with the highest concentrations of IGF-I (FIG. 15A, compare red and light blue tracings). Examination of individual cells from each of the latter two treatment groups revealed that the mean rate of nuclear import was enhanced up to 4-fold by inhibition of PI3-kinase, from ^{˜}3% to >12% per min (FIGS. 7B and 7C). Taken together, these results demonstrate that the FOXO1-clover reporter continually shuttles between nuclear and cytoplasmic compartments. Activation of Akt with IGF-I alters this equilibrium in favor of the cytoplasm, but does not prevent movement of the reporter into the nucleus, which was revealed when nuclear export was blocked with leptomycin B.

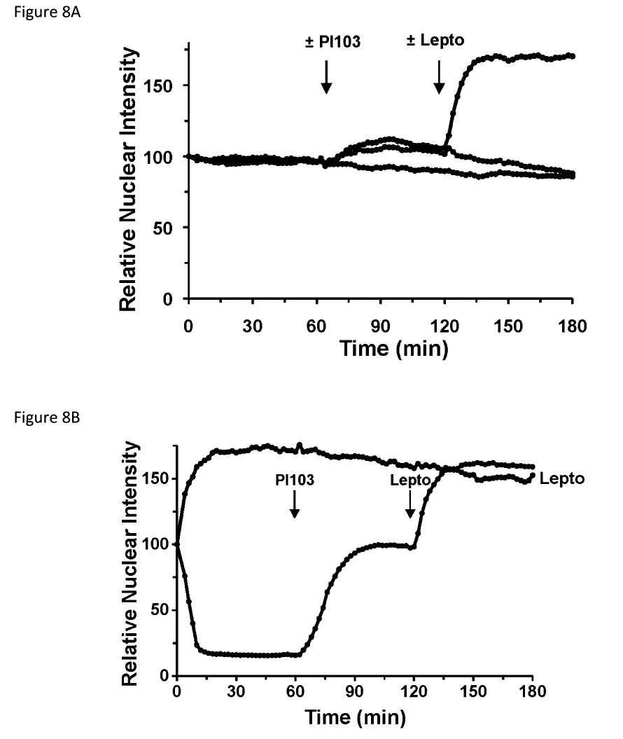

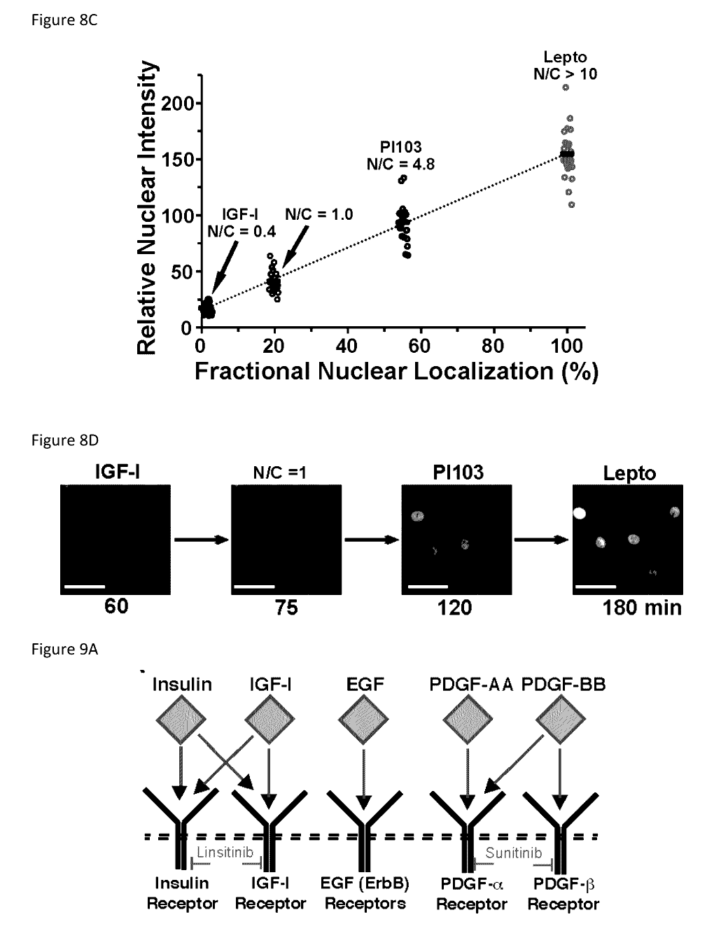

Incubation of cells with leptomycin B also showed that nuclear accumulation of the FOXO1-clover reporter could be increased significantly beyond the level seen in serum-free medium, raising the possibility that a basal level of Akt signaling was present even in cells that were not stimulated by serum or IGF-I. To address this question, cells were incubated in serum-free medium, followed by addition of PI103. As seen in FIG. 8A, PI103 caused a small increase (^{˜}10%) in the nuclear localization of the reporter compared with cells in serum-free medium alone (compare red and green tracings). Subsequent addition of leptomycin caused nearly a doubling of the nuclear intensity of the FOXO1-clover reporter (FIG. 8A). It can be concluded that in cells incubated in serum-free medium, there is little basal Akt activity.

Having established that exposure of cells to higher concentrations of IGF-I could promote extensive nuclear exclusion of the FOXO1-clover reporter protein, and conversely finding that leptomycin could maximize nuclear localization, a series of manipulations was performed to determine the actual fraction of reporter protein in the nucleus under different conditions. Nuclear and cytoplasmic fluorescence values for FOXO1-clover were measured at different time points during a sequential series of treatments: after serum starvation (time 0), at 60 min after incubation with IGF-I [250 pM], at 60 min after subsequent addition of PI103, and at 60 min after addition of leptomycin (summary population data appear in FIG. 8B and representative images in FIG. 8D). To place the observations in context with published studies using live-cell imaging (Regot S et al, Cell 157, 1724-1734 (2014); Tay et al, 2010 supra), at each time point also measured the ratio of nuclear to cytoplasmic fluorescence (N/C) was also measured, including when cytoplasmic and nuclear fluorescence intensities were identical (N/C=1). Although this varied among different cells, it typically occurred by ^{˜}15 min after addition of PI103 (FIGS. 8C, and 8D). To calculate the fraction of the FOXO1-clover reporter in each subcellular compartment, nuclear localization after 60 min of leptomycin treatment was pegged as 100% nuclear-localized, and 60 min of IGF-I [250 pM] as 100% cytoplasmic. With leptomycin, no cytoplasmic fluorescence was detected, but with IGF-I a small amount of nuclear fluorescence was detected, which was likely derived from cytoplasm being located above and/or below the nucleus in the cells analyzed. By fitting the values of cells incubated with PI103 and when N/C=1 between the two boundary conditions, we determined that ^{˜}56% of the reporter was in the nucleus after PI103 treatment and that ^{˜}19% was in the nucleus when the nuclear and cytoplasmic fluorescence intensities were equivalent (FIGS. 8C and 8D). It can be concluded from this analysis that nuclear to cytoplasmic ratios may be a misleading way to express data from live-cell imaging studies, as they may inaccurately estimate the true subcellular distribution.

### Example 5

### Materials and Methods

Reagents:

Fetal bovine serum (FBS) and newborn calf serum were obtained from Hyclone (Logan, Utah). Okadaic acid was from Alexis Biochemicals (San Diego, Calif.); protease inhibitor and NBT/BCIP tablets were purchased from Roche Applied Sciences (Indianapolis, Ind.). Dulbecco's modified Eagle's medium (DMEM), FluoroBrite, phosphate-buffered saline (PBS), and trypsin/EDTA solution were from Gibco-Life Technologies (Carlsbad, Calif.). Cells for imaging were grown on Greiner Bio-One tissue culture plates (Monroe, N.C.). Restriction enzymes, buffers, ligases, and polymerases were purchased from Roche Applied Sciences (Indianapolis, Ind.) and BD Biosciences-Clontech (Palo Alto, Calif.). AquaBlock EIA/WIB solution was from East Coast Biologicals (North Berwick, Me.). R3-IGF-I was purchased from GroPep (Adelaide, Australia), recombinant human PDGF-BB was from Invitrogen (Carlsbad, Calif.), and recombinant human BMP-2 purchased from R&D Systems (Minneapolis, Minn.). Growth factors were solubilized in 10 mM HCl with 1 mg/ml bovine serum albumin, stored in aliquots at −80° C., and diluted into FluoroBrite imaging medium immediately prior to use. The following primary antibodies were used: anti-Smad #H-465, Santa Cruz Biotechnology (Santa Cruz, Calif.), anti-phospho-Smad5^{Ser463+465} #76296, Abcam (Cambridge, United Kingdom), anti-Akt #4691, Cell Signaling (Beverly, Mass.), anti-phospho-Akt^{Thr308} #2965, Cell Signaling, and anti-α-tubulin, Sigma-Aldrich (St. Louis, Mo.). Secondary antibodies included goat anti-rabbit and anti-mouse IgG conjugated with Alexa Fluor 680 (Invitrogen), and IR800-conjugated goat anti-rabbit IgG, Rockland (Gilbertsville, Pa.). Puromycin was purchased from Enzo Life Sciences (Farmingdale, N.Y.), polybrene was from Sigma-Aldrich, and leptomycin B was from Cell Signaling ([200 μM] solution in ethanol). PI103 was from Tocris (Bristol, United Kingdom), and was solubilized in DMSO. Other chemicals and reagents were purchased from commercial suppliers.

Production of Recombinant Lentiviruses:

To construct a recombinant lentivirus encoding the FOXO1-clover fusion protein, a cDNA for full-length mouse FOXO1 was generated by PCR, using the cDNA insert from pdsRED-Mono-N1-FOXO1 as a template (plasmid #34678, Addgene, Cambridge, Mass.). The 3′ end of the FOXO1 coding region was ligated in-frame to the 5′ end of the green fluorescent protein, clover (Lam et al 2012 supra). The following three amino acid substitutions were introduced into the DNA of the Forkhead domain of FOXO1, using splice-overlap-extension PCR: S212A, H215R, and S218A. All DNA modifications were confirmed by sequencing. Recombinant lentiviruses were prepared by co-transfecting a transfer vector containing the FOXO1-clover cDNA with third-generation packaging plasmids (#12251, #12253, #12259, Addgene) into Hek293FT cells (Gibco-Life Technologies) as described (Tiscornia G et al, Nat Protoc 1, 241-245 (2006); incorporated by reference herein). Virus was purified and concentrated by centrifugation of cell culture supernatant at 19,000×g at 4° C. for 2 hours (Mukherjee et al, 2010 infra).

Lentiviral Infection and Selection:

C3H10T1/2 mouse embryonic fibroblasts (ATCC #CCL226) were incubated in DMEM supplemented with 10% FBS. Mouse C2 myoblasts (Yaffe and Saxel, 1977) were grown in DMEM supplemented with 10% FBS and 10% newborn calf serum. Cells were transduced at 50% of confluent density with concentrated virus in the presence of 6 μg/ml polybrene, as described (Mukherjee A et al, Mol Cell Biol 30, 1018-1027 (2010); incorporated by reference herein). Cells were then selected by incubation with puromycin (2 μg/ml) for one week. Surviving cells were sorted by fluorescence intensity using a Becton-Dickinson Influx cell sorter at the OHSU Flow Cytometry Core Facility. Reporter expression was stable for at least 10 passages in each sorted cell population.

Long-Term Imaging Under Cellular Growth Conditions:

10T1/2 cells were imaged every 10-min for ^{˜}16 hours in supplemented FluoroBrite medium plus 10% FBS. Cells were then washed twice with DMEM and incubated for 90 min in serum-free supplemented FluoroBrite.

Responses to Different Growth Factors:

10T1/2 cells were incubated in supplemented FluoroBrite plus R3-IGF1 [1 nM], PDGF-BB [206 pM], BMP-2 [15 nM]), 10% FBS, or vehicle. Cells were imaged every 2-min for 60 min. At the end of the incubation period whole cell lysates were collected.

Responses to IGF-I:

10T1/2 cells and C2 myoblasts were incubated in serum-free medium for 90 min. R3-IGF-I was added in supplemented FluoroBrite [0 to 500 pM], and cells were imaged every 2-min for 60 min.

Effects of Leptomycin and PI3-Kinase Inhibition:

10T1/2 cells were incubated in serum-free medium for 90 min. R3-IGF-1 was added in supplemented FluoroBrite [at 0 to 500 pM, see Figure Legends 7 and 8], and cells were imaged every 2-min. After 60 min, medium was supplemented with leptomycin B [100 nM], PI103 [500 nM], or both drugs, and imaging was continued for up to another 120 min. Kinetics of nuclear export were calculated by fitting individual cell responses to a single exponential equation using GraphPad Prism (San Diego, Calif.).

Imaging Data Analysis.

To assess signaling variability over time in cells incubated in 10% FBS, measurements of nuclear intensity of the FOXO1-clover reporter were summed from each of 50 cells for 4 hrs (total of 24 data points per cell) using information from FIG. 2B, and the mean value was determined for each cell. The absolute deviation from the mean was then calculated at each time point, and across all time points. To assess measurement error, the nuclear intensity of the FOXO1-clover reporter was determined in each of 5 cells 10 times by analyzing the same video recordings. These results were summed and the average absolute deviation was calculated. Statistical significance was calculated using an unpaired Student's t test for FIG. 7C. To determine the fraction of the FOXO1-clover reporter in the nucleus in FIG. 8C, fluorescence intensities were measured in the nuclear and cytoplasmic compartments of 10T1/2 cells treated with R3-IGF-I [250 pM], PI103 [500 nM], and leptomycin [100 nM] at 5 different time points: (1) in serum-free medium; (2) after 60 min of IGF-I; (3) when nuclear fluorescence in the nucleus and peri-nuclear cytoplasm were equal (this time point varied, but usually occurred ^{˜}15 min after the addition of PI103; (4) 60 min after addition of PI103; (5) 60 min after addition of leptomycin. For subsequent quantification, the nuclear fluorescence intensity at 60 min after IGF-I treatment was assigned a value of 0% nuclear localization, and intensity at 60 min after leptomycin was assigned a value of 100% nuclear localization. From these two values, we constructed a linear equation to calculate the percent nuclear localization based on the nuclear fluorescence intensity. We used this equation to calculate the percent nuclear localization after incubation of cells with PI103, and when nuclear and cytoplasmic fluorescence intensities were equivalent (see FIG. 8C).

Protein Extraction and Immunoblotting.

Whole cell protein lysates were prepared as described (Mukherjee A and Rotwein P, Mol Endorcrinol 22, 1238-1250 (2008); incorporated by reference herein). Protein aliquots (15 μg/lane) were resolved by SDS-PAGE (12% separating gel), followed by transfer to Immobilon-FL membranes, and blocking with 50% AquaBlock solution. Membranes were incubated sequentially with primary and secondary antibodies, as described (Mukherjee and Rotwein, 2008 supra). Primary antibodies were incubated for 12-16 hr at a 1:1000 dilution, except for α-tubulin (1:10,000), and secondary antibodies for 90 min at 1:5000. Images were captured using the LiCoR Odyssey and version 3.0 analysis software (Lincoln, Nebr.).

### Example 6

### Mapping Growth Factor Encoded Akt Signaling Dynamics

Growth factors alter cellular behavior through shared signaling cascades, raising the question of how specificity is achieved. Disclosed herein is how growth factor actions are encoded into Akt signaling dynamics by real-time tracking of a fluorescent sensor. In individual cells, Akt activity was encoded in an analog pattern, with similar latencies (^{˜}2 min) and half-maximal peak response times (^{˜}6±2 min). Yet, different growth factors promoted dose-dependent and heterogeneous changes in signaling dynamics. Insulin treatment caused sustained Akt activity, while EGF or PDGF-AA promoted transient signaling; PDGF-BB produced sustained responses at higher concentrations, but short-term effects at low doses, actions that were independent of the PDGF-α receptor. Transient responses to EGF were caused by negative feedback at the receptor level, as a second treatment yielded minimal responses, while parallel exposure to IGF-I caused full Akt activation. Small molecule inhibitors reduced PDGF-BB signaling to transient responses, but only decreased the magnitude of IGF-I actions. Our observations reveal distinctions among growth factors that use shared components, and allow us to capture the consequences of receptor-specific regulatory mechanisms on Akt signaling.

Cells interpret their local environment by encoding extracellular cues into intracellular signaling responses. Peptide growth factors are one class of extracellular molecules that stimulate signaling pathways to control cell growth, proliferation, or metabolism (Cross M and Dexter T M, Cell 64, 271-280 (1991); incorporated by reference herein). Each of these peptides typically binds and activates a distinct subset of trans-membrane receptors, thereby regulating a variety of intracellular signaling cascades (Lemmon M A and Schlessinger J, Cell 141, 1117-1134 (2010); incorporated by reference herein). The biochemical steps downstream of each receptor are shared among several classes of growth factors and have been relatively well defined (Lemmon and Schlessinger 2010 supra; Manning B D and Cantley L C, Cell 129, 1261-1274 (2007); incorporated by reference herein.) Yet, different growth factors induce distinctive behavioral responses in cells (Downward J, Nature 411, 759-762 (2001) and Marshall M, Mol Reprod Dev 42, 493-499 (1995); incorporated by reference herein), suggesting that variability in signaling dynamics or other related processes may be key determinants in producing unique biological outcomes.

Previous analyses of signaling dynamics downstream of growth factor receptors have led to several different observations and initial conclusions. For example, PDGF-BB has been found to promote graded short-term activation of the PI3-kinase-Akt pathway (Park C S et al, J Biol Chem 278, 37064-37072 (2003); incorporated by reference herein), with signaling diminishing over extended time periods (Cirit M and Haugh J M, Biochem J 441, 77-85 (2012); incorporated by reference herein). By contrast, insulin has been shown to lead to transient or sustained Akt signaling responses (Kubota H et al, Mol Cell 46, 820-832 (2012); incorporated by reference herein, as has EGF (Borisov N et al, Mol Syst Biol 5, 256 (2009), Chen W W et al, Mol Syst Biol 5, 239 (2009); both of which are incorporated by reference herein). In general, as these results have been based on end point assays that measure mean responses of a population, the data may not accurately reflect behavior at the single cell level. Additionally, many of these studies did not evaluate the actions of different growth factors in the same cellular context, thus leaving analyses incomplete.

Recently, fluorescent reporter molecules have been developed to track signaling pathways in real time (Purvis J E and Lahav G, Mol Cell 46, 715-716 (2012), Regot S et al, Cell 157, 1724-1734 (2014); and Yissachar N et al, Mol Cell 49, 322-330 (2013); all of which are incorporated by reference herein). Results generated by these approaches, which have included FRET-based reporters and other strategies, have shown not only that signaling dynamics of individual cells tend to be hidden within population averages, but also that some pathways yielded sustained responses, others produced transient effects, and still others showed variable patterns depending upon either the strength or duration of the signaling input (Albeck J G et al, Mol Cell 49, 249-261 (2013); and Batchelor E et al, Mol Syst Biol 7, 488 (2011); both of which are incorporated by reference herein). Some signaling pathways also have been found to exhibit all-or-none (=digital) outcomes (Tay S et al, Nature 455, 267-271 (2010); incorporated by reference herein), while others have demonstrated graded (=analog) responses (Toettcher J E et al, Cell 155, 1422-1434 (2013); incorporated by reference herein). It thus has become apparent that population averages and endpoint assays provide at best a limited understanding of overall cellular signaling behavior.

Disclosed herein is a fluorescent reporter protein based on the FoxO1 transcription factor that rapidly and robustly transited from the nucleus to the cytoplasm in response to stimulation of Akt kinase activity (Gross S M and Rotwein P, J Cell Sci 128, 2509-2519 (2015); incorporated by reference herein). With this sensor, the dynamics of Akt activity could be quantified over short and longer time courses, and it was found that IGF-I-mediated Akt signaling was encoded into stable and reproducible analog responses at the population level, but that Akt signaling outputs were highly variable among individual cells, particularly after exposure to low growth factor concentrations.

Further, Akt signaling dynamics in response to treatment of cells with four different growth factors has been evaluated. The disclosed results provide a quantitative experimental platform for determining how growth factors regulate cellular behavior and reveal the complex nature of how signaling pathways are encoded into different cellular outcomes.

Growth Factors and Akt Activity:

A fluorescent reporter protein designed to assess Akt activity at the single cell level is described herein. The reporter comprises a fusion of the green fluorescent protein, clover (Lam et al, 2012 supra), to the COOH-terminus of FoxO1, a well-characterized Akt substrate (Brunet et al, 1999 supra; Rena et al, 1999 supra; Rena et al, 2002 supra; Zhang et al, 2002 supra). We modified the FoxO1 portion of the chimeric molecule to inhibit its DNA binding activity (Tang et al, 1999 supra), and to prevent effects of phosphorylation by the Mst1 protein kinase (Lehtinen et al, 2006 supra). After lentiviral delivery into cells, stable selection, and cell sorting, rapid and robust reporter transit from the nucleus to the cytoplasm in response to exposure to serum or to the growth factor, IGF-I was visualized.

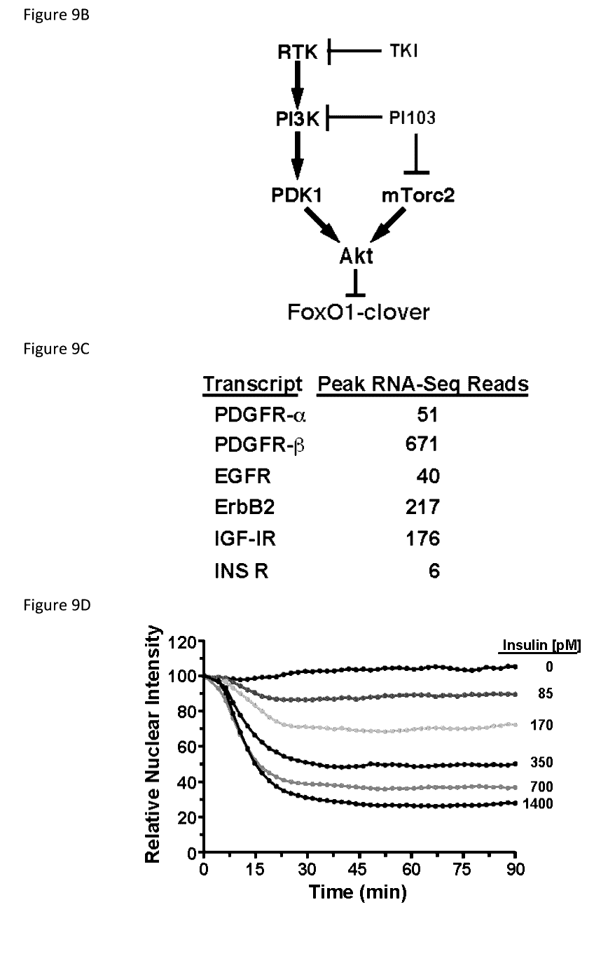

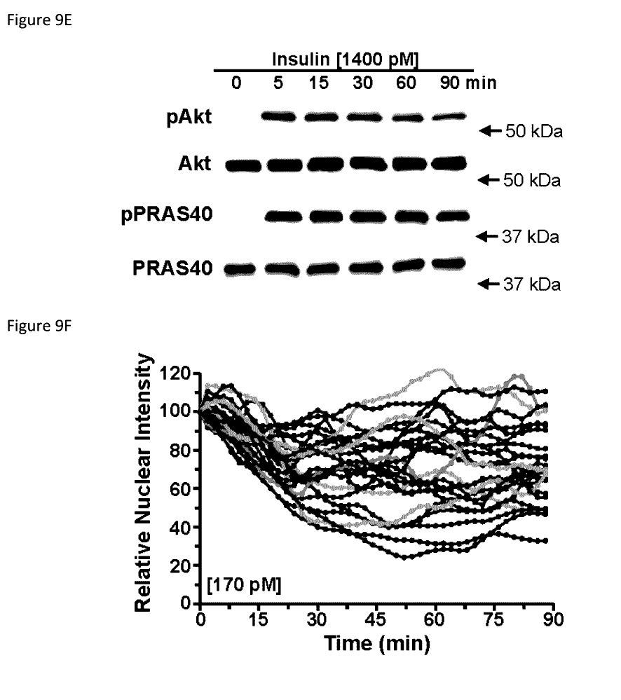

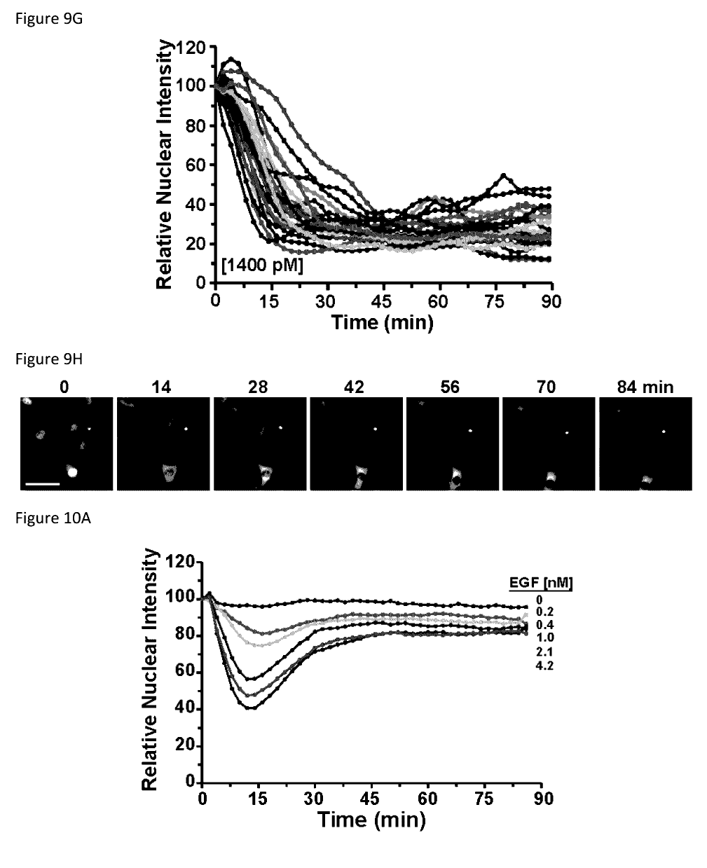

In order to test how other growth factors regulate Akt signaling activity, the same C3H10T1/2 cells were treated with varying concentrations of insulin, EGF, PDGF-AA, or PDGF-BB, and real-time responses were monitored by live-cell imaging. Each of the growth factors tested engages a ligand-stimulated tyrosine kinase receptor (FIG. 9A), and activates the PI3-kinase-Akt signaling pathway (FIG. 9B), but through different intermediary molecules (Lemmon and Schlessinger, 2010 supra). Published RNA-seq data from C3H10T1/2 cells showed that mRNA encoding receptors for each growth factor are expressed, but at different steady-state levels (FIG. 9C).

Cells Respond in a Graded and Sustained Manner to Insulin:

The hormone insulin binds both to the insulin receptor and to the IGF-I receptor, although with ^{˜}1000 fold less affinity for the latter (Blakesley V A et al, Cytokine Growth Factor Rev 7, 153-159 (1996); incorporated by reference herein). As found previously, the FoxO1-clover reporter protein was predominantly nuclear in cells incubated in serum-free medium (SFM) (FIGS. 9D, 9F-9H). Addition of insulin caused a rapid, dose-dependent, and sustained decrease in nuclear levels of FoxO1-clover, with half-maximal accumulation in the cytoplasm being seen by 8-15 min after onset of incubation, and maximal values being attained by ^{˜}20 min (FIG. 9D).

To ascertain if the reporter was tracking Akt activity, both Akt phosphorylation and the phosphorylation of another Akt substrate, PRAS40 were serially measured, by immunoblotting whole cell protein lysates from cells treated with the highest dose of insulin [1400 pM]. Phosphorylation of Akt and PRAS40 were each rapid and sustained, being detected within 5 min of insulin exposure and being maintained over the entire 90 min observation period (FIG. 9E) These results are similar to those observed with the FoxO1-clover reporter by live-cell imaging (FIGS. 9D and 9H).

The time-course studies and immunoblotting results in FIGS. 9C and 9D represent population averages, and do not provide insight into the behavior of individual cells exposed to different hormone concentrations. So the single cell data from which the population averages were derived was examined and it was found that responses to insulin were variable, particularly at lower hormone concentrations [170 pM] (FIG. 9F). At higher insulin exposures [1400 pM], initial rates and the extent of export of the FoxO1-clover reporter from the nucleus were more substantial, and less heterogeneous than at lower hormone concentrations (FIG. 9G). Taken together the results in FIGS. 9A-9G show that like IGF-I, effects of a given concentration of insulin on individual cells are graded, with exposure to higher hormone levels leading to more sustained and less variable outcomes than lower concentrations.

Cells Respond Transiently to EGF:

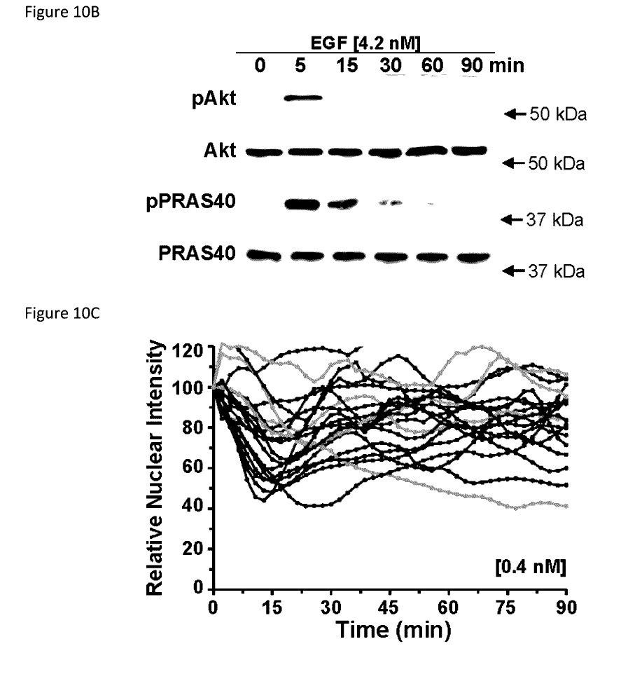

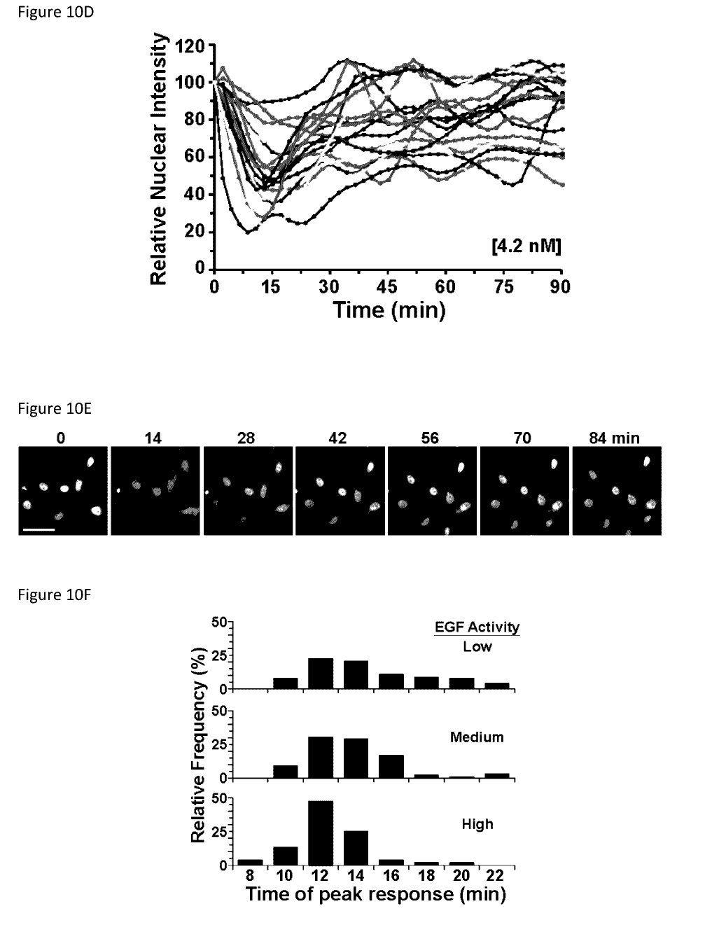

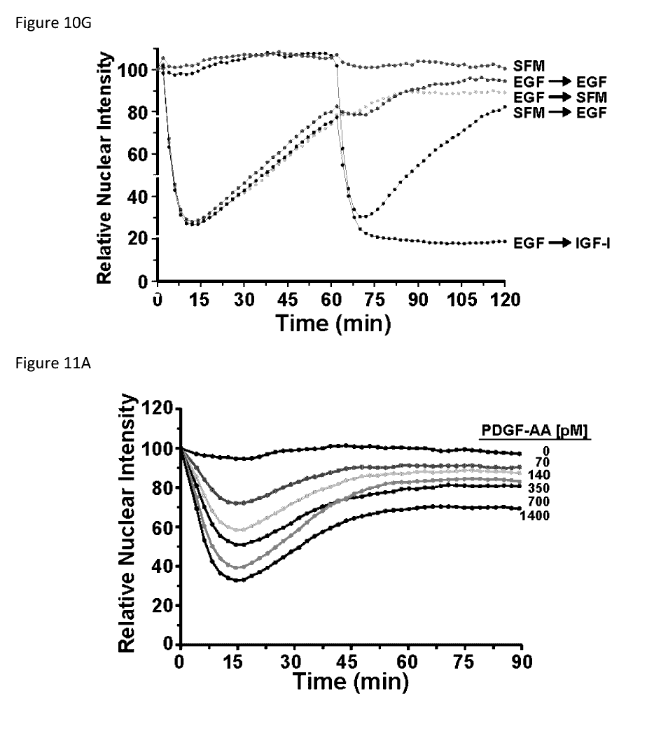

To test if graded and sustained responses are the standard pattern for how growth factor signals are encoded into Akt activity, cells were next exposed to different concentrations of EGF. EGF-mediated signaling is complicated because the growth factor can bind to any of three receptors, EGFR (ErbB1), ErbB3, or ErbB4, but with different affinities (Citri A and Yarden Y, Nat Rev Mol Cell Biol 7, 505-516 (2006); Riese D J et al, Bioessays 29, 558-565 (2007); incorporated by reference herein), leading to a variety of homo- and heterodimers, including those containing ErbB2, which lacks growth factor binding capabilities. Addition of EGF to cells pre-incubated in SFM caused rapid, dose-dependent, and transient decreases in nuclear levels of the FoxO1-clover reporter protein. At the population level, half-maximal accumulation of the reporter in the cytoplasm was observed by 6-9 min after EGF treatment, with maximal values being reached by ^{˜}13 min, and signal intensity waning by 45 min (FIG. 10A). Similarly transient effects were seen by immunoblotting of treated cells for Akt or PRAS40 phosphorylation (FIG. 10B), and also were observed at the single cell level, where growth factor-160 mediated signaling was found to be highly heterogeneous at both lower and higher EGF concentrations (FIGS. 10C-10F). As noted in FIG. 10F, the timing of responses of individual cells to EGF in terms of nuclear to cytoplasmic translocation of the FoxO1-clover reporter molecule was fairly similar, even when stratified by overall EGF activity, although a larger fraction of cells reached a peak earlier compared to cells exhibiting a lower maximal response.

It was then asked if these transient signaling responses to EGF were caused by a negative feedback loop that inhibited Akt signaling at the level of PI3-kinase or further downstream, and thus would prevent Akt activation by another growth factor. To address this question, cells were first incubated with EGF [4.2 nM] for 60 min, followed by the addition of EGF [4.2 nM] or IGF-I [500 pM]. We found that a second EGF treatment minimally promoted FoxO1-clover reporter translocation out of the nucleus (FIG. 10G). In contrast, addition of IGF-I caused a rapid, extensive, and sustained signaling response (FIG. 10G). These results show that negative feedback of EGF-mediated signaling in C3H10T1/2 cells is located upstream of the PI3 kinase-Akt module, and likely resides at the level of the receptor.

Variable Responses of the FoxO1-Clover Reporter Protein to PDGF-AA or PDGF-BB:

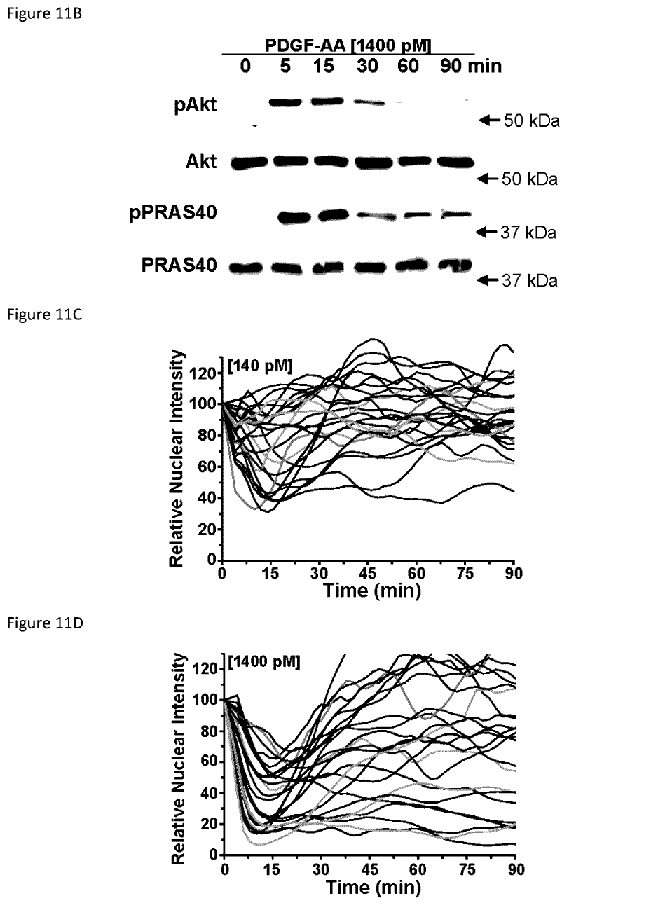

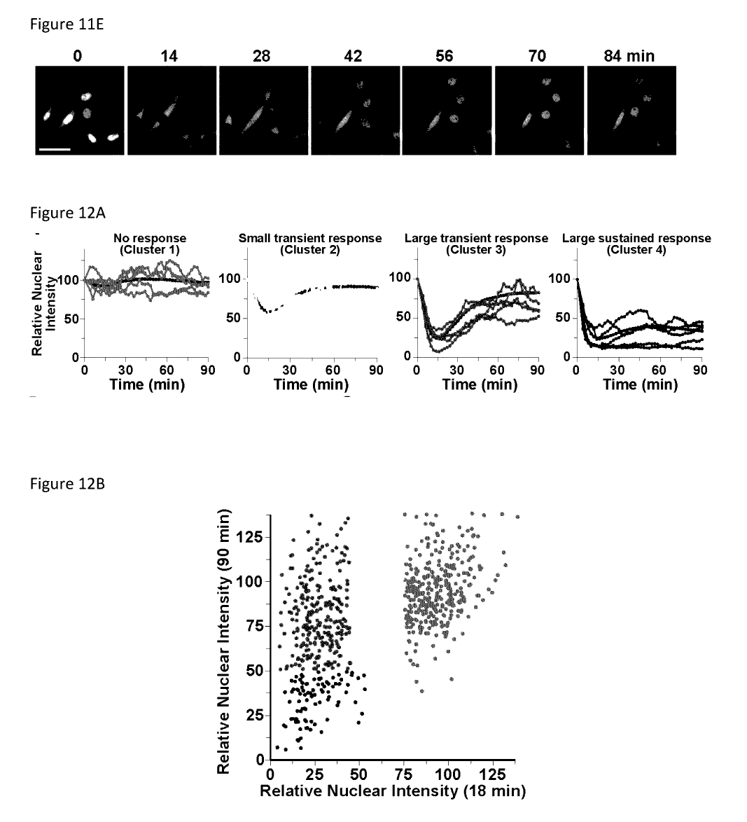

Cells were then exposed to different concentrations of PDGF-AA and PDGF-BB. These two growth factors function as dimers, and vary in their affinity for PDGF-α and PDGF-β receptors (Andrae J et al, Genes Dev 22, 1276-1312 (2008); incorporated by reference herein. PDGF-AA binds almost exclusively to the PDGF-α receptor, whereas PDGF-BB binds to both receptors (FIG. 9A). Cells incubated with PDGF-AA showed rapid, dose-dependent decreases in nuclear levels of FoxO1-clover, with half-maximal accumulation in the cytoplasm by 6-10 min, and maximal values by ^{˜}14 min (FIG. 11A). In contrast to the effects of insulin, but similar to EGF, population responses to PDGF-AA were transient, as they declined by 50-75% from peak values over the next 40 min (FIG. 11A). Similarly brief effects were seen for PDGF-AA-stimulated phosphorylation of Akt and PRAS40, as measured by immunoblotting in cells incubated with the highest growth factor concentration [1400 pM] (FIG. 11B).

Analysis of single cell data obtained by live-cell imaging revealed that individual responses to PDGF-AA were highly variable. At both lower [140 pM] and higher growth factor concentrations [1400 pM], the effects of PDGF-AA ranged from minimal and transient to substantial and sustained (FIGS. 11C-11E). These results illustrate that population averages serve as a poor proxy for signaling responses to PDGF-AA at the single cell level.

To further quantify the effects of PDGF-AA on individual cells, all of the single cell responses were clustered into four distinct groups based on signaling dynamics, using relative nuclear intensity of the FoxO1-clover reporter at 18 and 90 min time points as a guide. The results were grouped as showing no response, a small transient effect, a larger transient response, or large and sustained effects (FIGS. 12A, 12B). When these results were graphed against PDGF-AA concentration, it can be seen that as the growth factor dose rose, the fraction of cells demonstrating more extensive responses increased (FIG. 12C). However, even at the two highest concentrations of PDGF-AA, 25-30% of cells showed no or minimal responses, and only 10-15% demonstrated sustained effects (FIG. 12C).

The signaling dynamics of cells treated with PDGF-BB were different from those incubated with PDGF-AA. At lower growth factor concentrations, the mean response of the FoxO1-clover reporter was transient and resembled effects of PDGF-AA, with half-maximal accumulation in the cytoplasm by 8-9 min, and maximal values by ^{˜}14 min (FIG. 13A). In contrast, at higher concentrations of PDGF-BB, signaling responses were more rapid and extensive, as half-maximal accumulation of FoxO1-clover in the cytoplasm was seen by <5 min, and cytoplasmic localization of the reporter was maintained for at least 90 min (FIG. 13A). Similarly sustained signaling was seen in immunoblots of Akt and PRAS40 phosphorylation after exposure of cells to highest concentrations of PDGF-BB [104 pM] (FIG. 13B).

Analysis of individual cells confirmed the dose-dependent heterogeneity of signaling responses to PDGF-BB. At low growth factor concentrations [5.2 pM] effects on individual cells were highly variable, with some cells showing no changes in the nuclear localization of the FoxO1-clover reporter, and others maintaining sustained cytoplasmic translocation (FIG. 13C). In contrast, at the highest levels of growth factor exposure [104 pM], the effects of PDGF-BB were similar to those seen with the highest concentrations of insulin, as the reporter was rapidly trans-located to the cytoplasm in nearly all cells, and was maintained there for the 90 min duration of the experiment (FIGS. 13D, 13E). When individual cellular responses to PDGF-BB were graphed using the same criteria as for PDGF-AA, results also showed dose-dependent effects ranging from small and transient to large and sustained signaling, but with sharper and more complete transitions than were observed for PDGF-AA: at peak concentrations of PDGF-BB, 90-99% of cells showed large and sustained signaling responses (FIGS. 13F, 13G).

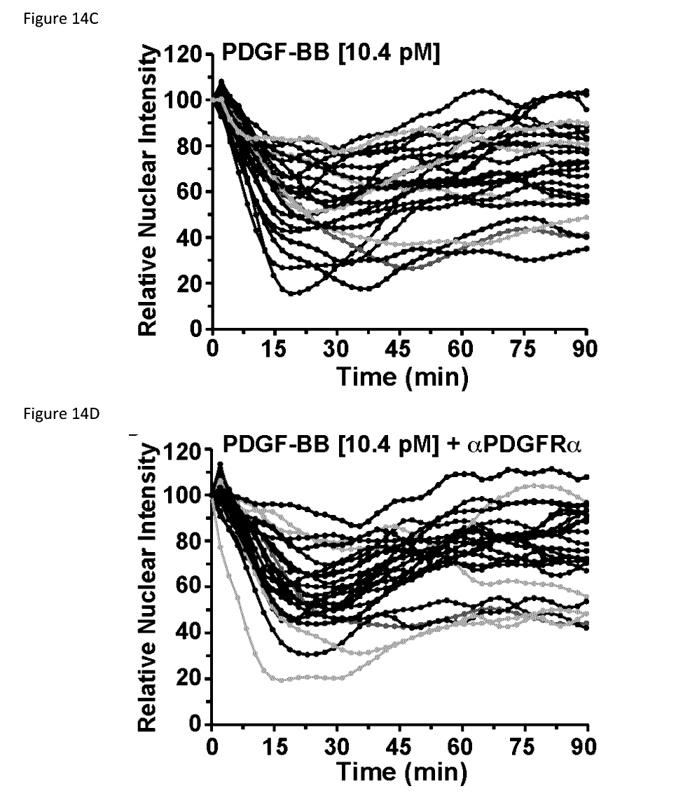

The signaling dynamics initiated by PDGF-BB reflect combined engagement of both PDGF-α and PDGF-β receptors, while effects of PDGF-AA are mediated solely by PDGF-α receptors. To identify signaling exclusively through PDGF-β receptors, cells were incubated with a neutralizing antibody to PDGF-α. In the presence of antibody, signaling by PDGF-AA was completely inhibited, while exposure of cells to control IgG had no effect (FIG. 14A). In contrast, the same anti-PDGF-α antibody reduced responses to sub-maximal concentrations of PDGF-BB by only ^{˜}10%, had no effect at the highest PDGF-BB dose (FIG. 14B), and did not alter single cell dynamics (FIG. 14C). Taken together, these results demonstrate remarkable plasticity in PDGF-mediated signaling dynamics for Akt that appear to be dependent on the type and number of PDGF receptors being activated.

Chemical Inhibitors Recapitulate Dose-Dependent PDGF-BB Signaling:

Signaling by PDGF-BB becomes greater in magnitude and and more sustained as cells are exposed to higher growth factor concentrations. To understand the mechanisms behind this process and to learn how the downstream Akt signaling pathway is wired, cells were incubated with different amounts of the PDGF receptor tyrosine kinase inhibitor, Sunitinib, in the presence of high concentrations of PDGF-BB. Under these conditions, Sunitinib caused a dose-dependent shift from sustained to more transient Akt activity, as measured by the subcellular location of the FoxO1-clover reporter (FIG. 15A). Similar results were observed with the dual PI3-kinase and mTor inhibitor, PI103, which more directly blocks Akt signaling (FIG. 15B). Thus, it appears that inhibiting either the receptor or downstream pathway activity results in recapitulating signaling dynamics from submaximal PDGF-BB stimulation.

To learn more generally if inhibition of different components of a signaling pathway results in comparable effects, cells were exposed to graded concentrations of the IGF-I-insulin receptor specific tyrosine kinase inhibitor, Linsitinib, or to PI103. In contrast to results with PDGF-BB, each inhibitor caused a dose-dependent decline in maximal IGF-I-mediated cytoplasmic localization of the FoxO1-clover reporter molecule, but did not reduce the duration of signaling (FIGS. 15C, 15D). Moreover, PI103 was more effective in blocking PDGF-BB-stimulated Akt activity than in blunting IGF-I actions, as the IC₅₀ for PDGF-BB-mediated signaling was between 20 and 50 nM, while for IGF-I it was between 50 and 200 nM (compare FIGS. 15B and 15D). These results show that the same downstream inhibitor can reveal variability of signaling responses initiated by different upstream activators.

General Variability in Growth Factor Signaling Dynamics:

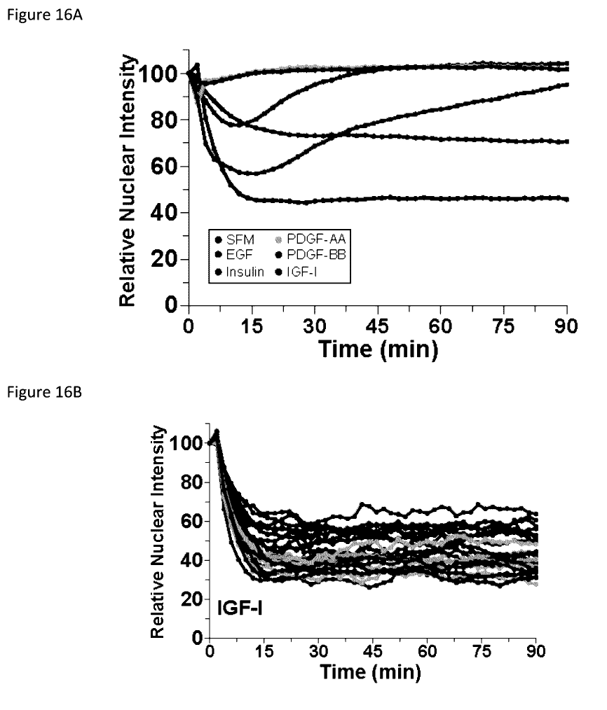

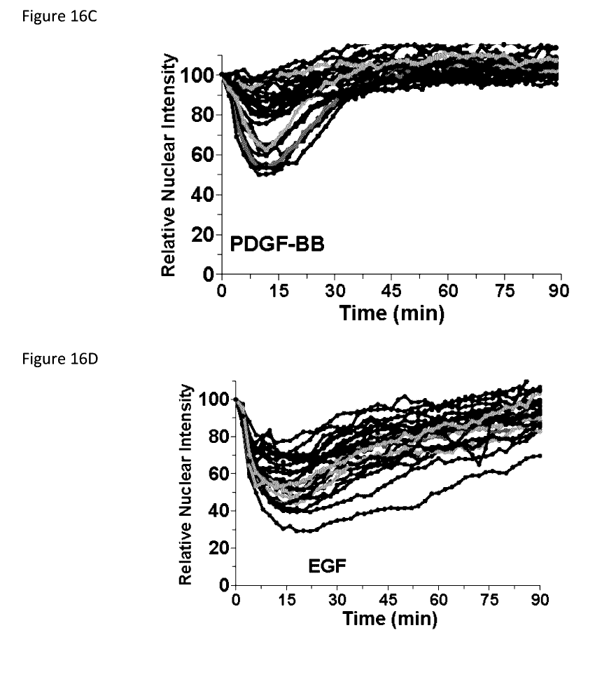

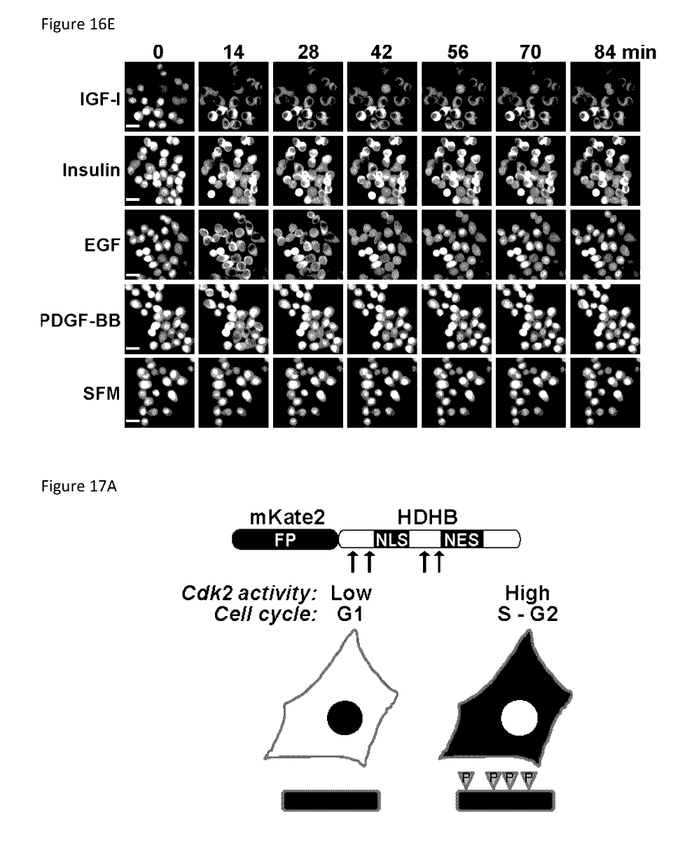

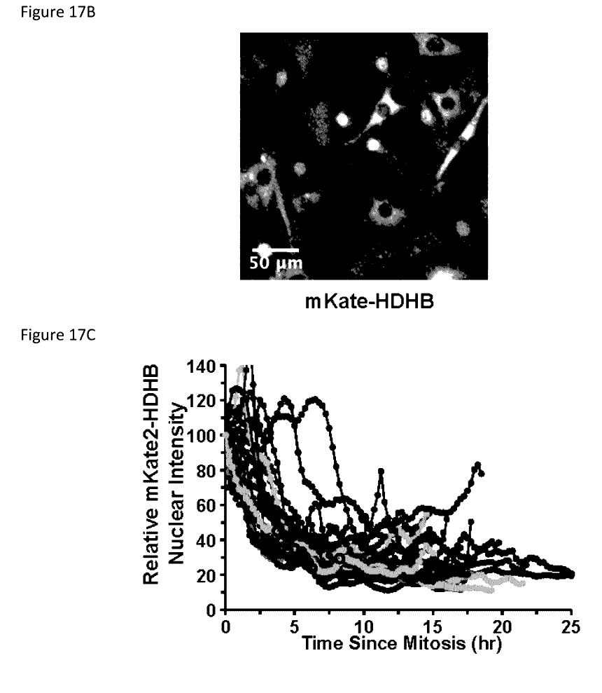

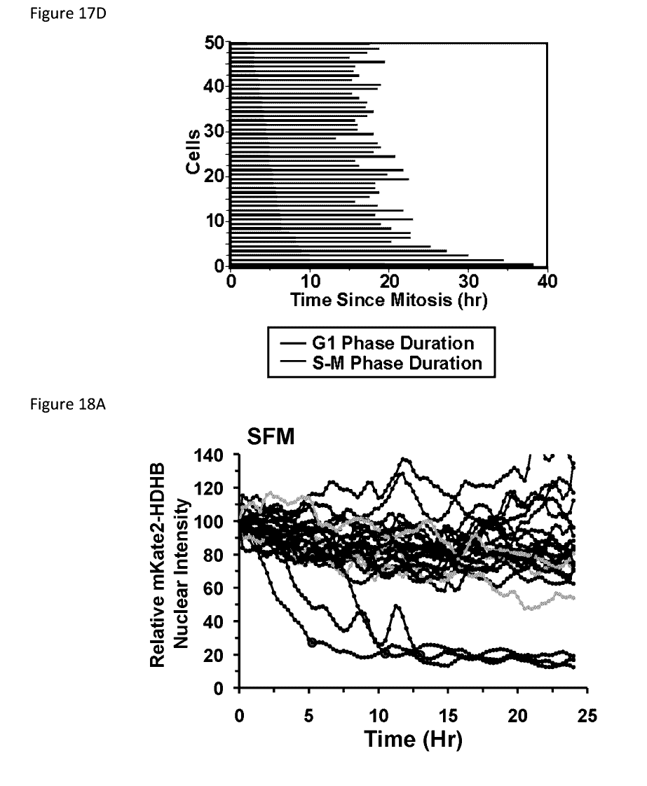

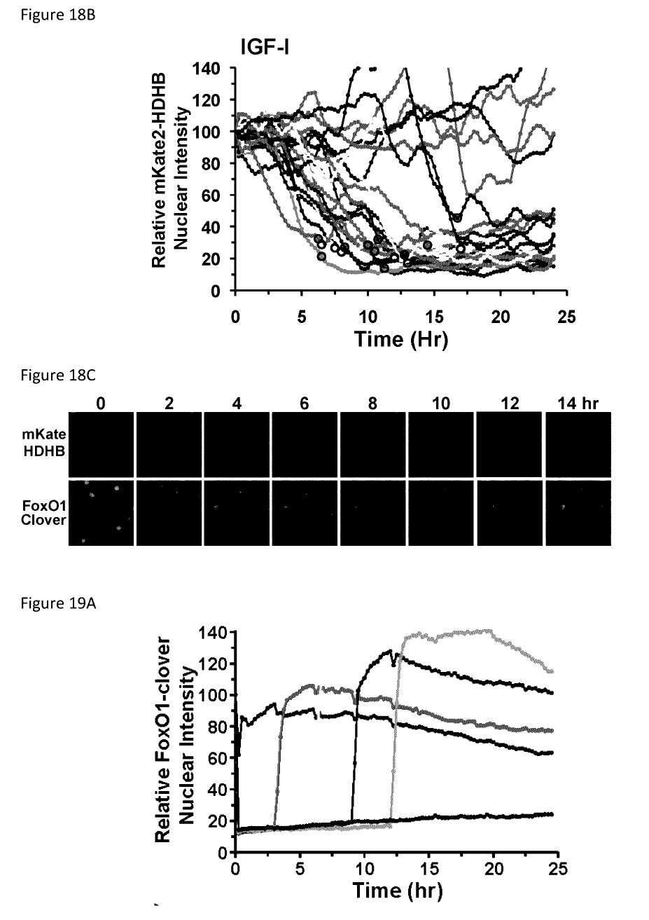

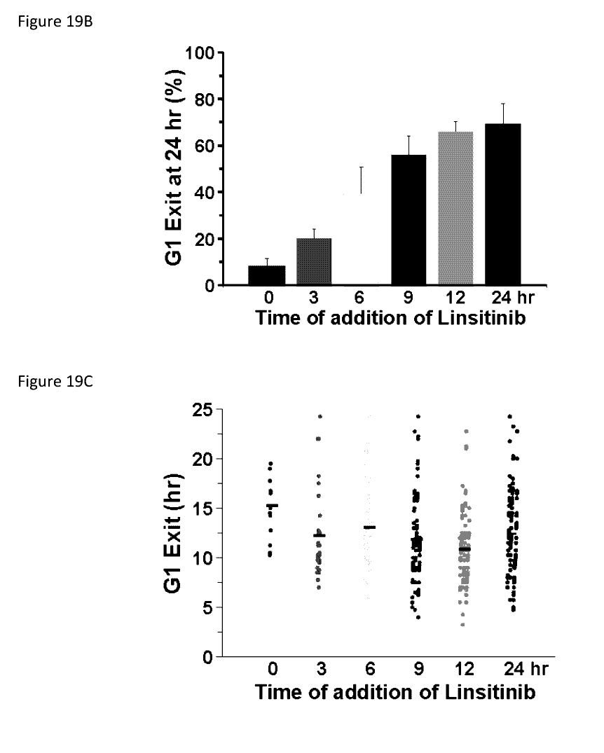

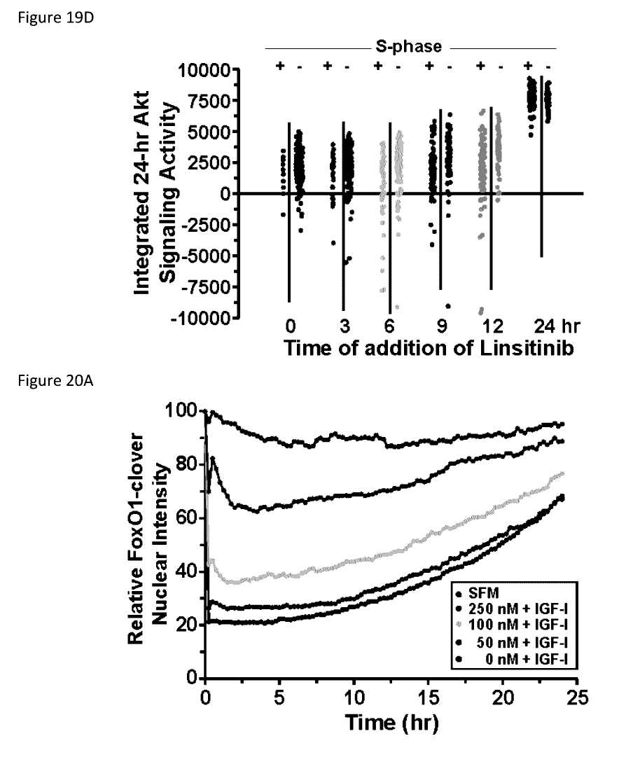

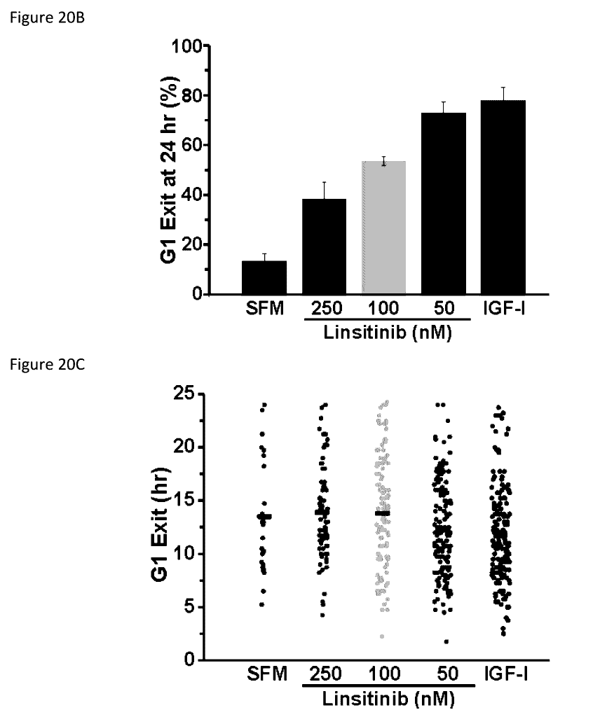

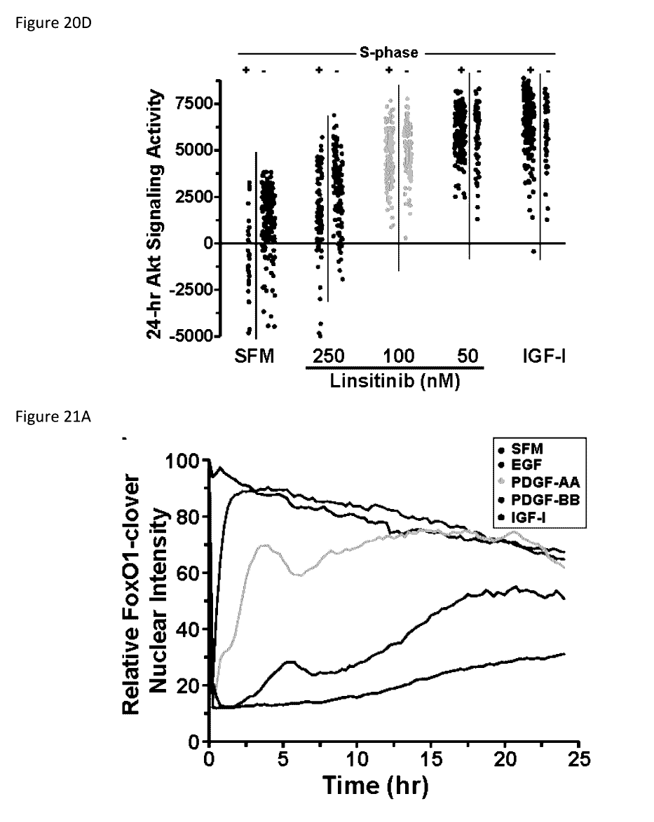

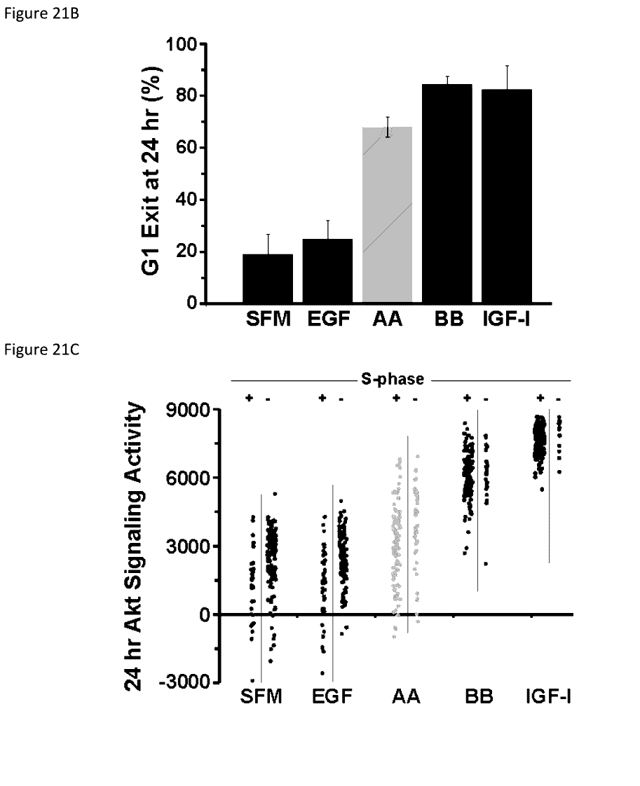

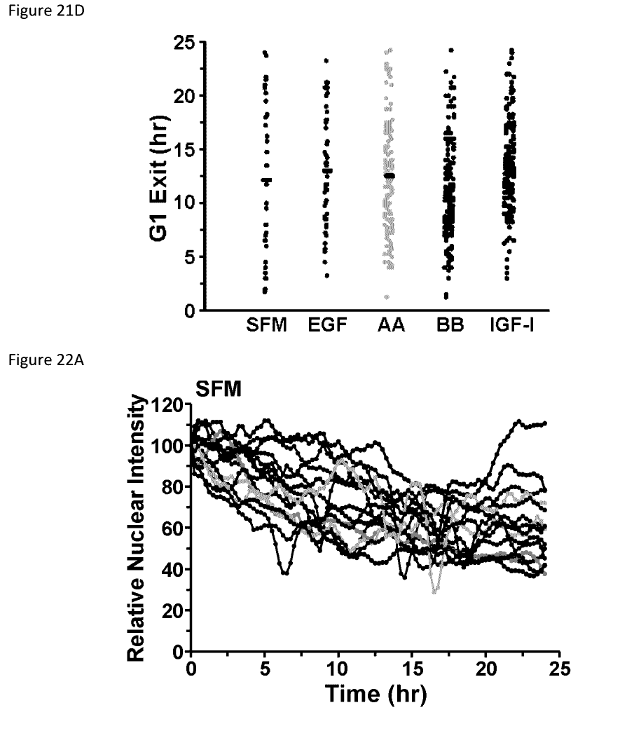

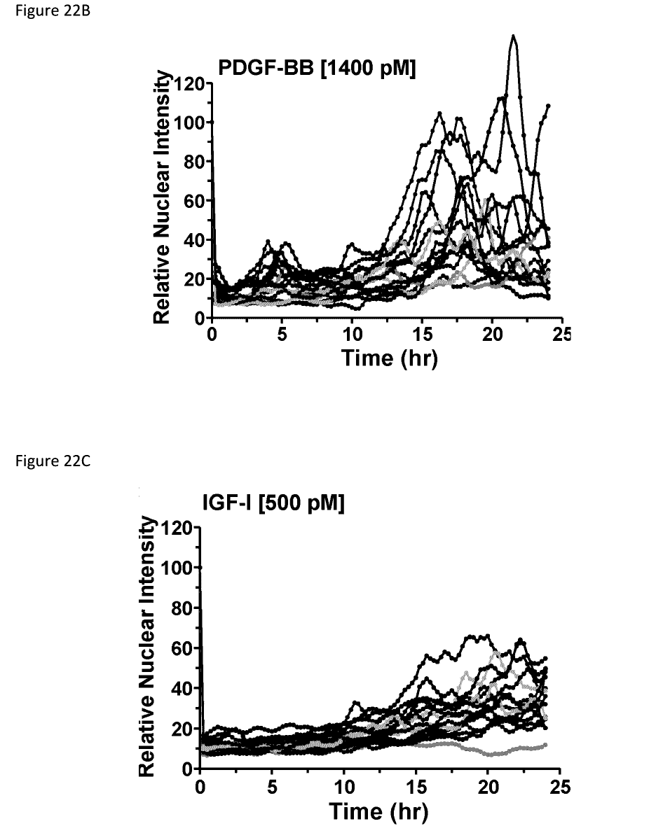

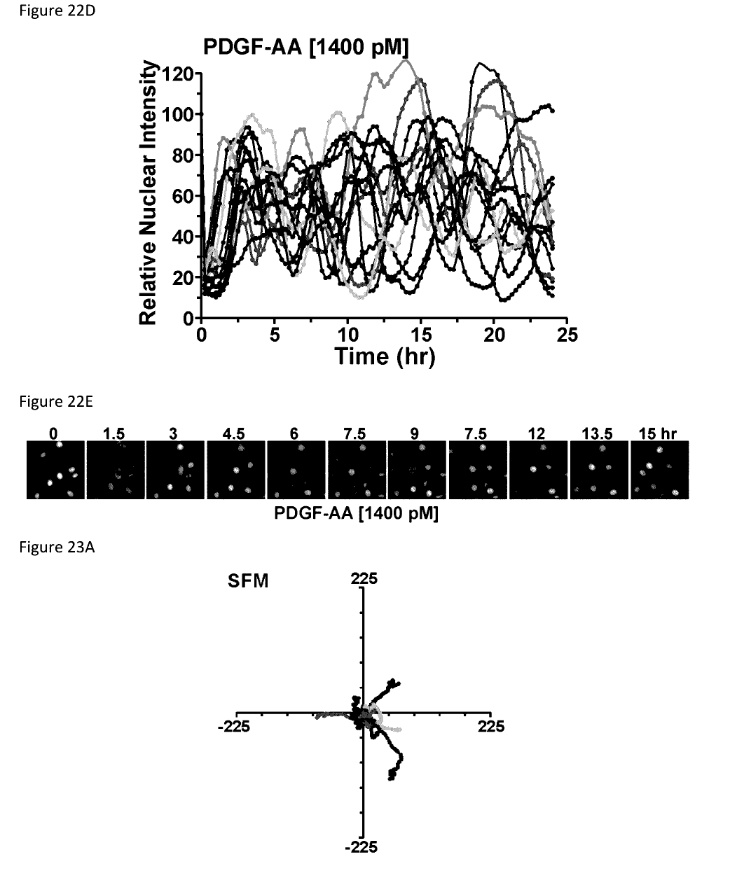

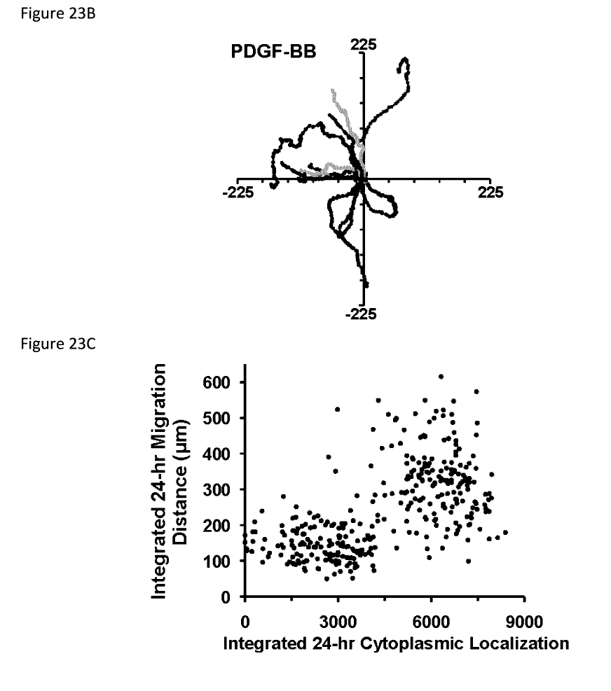

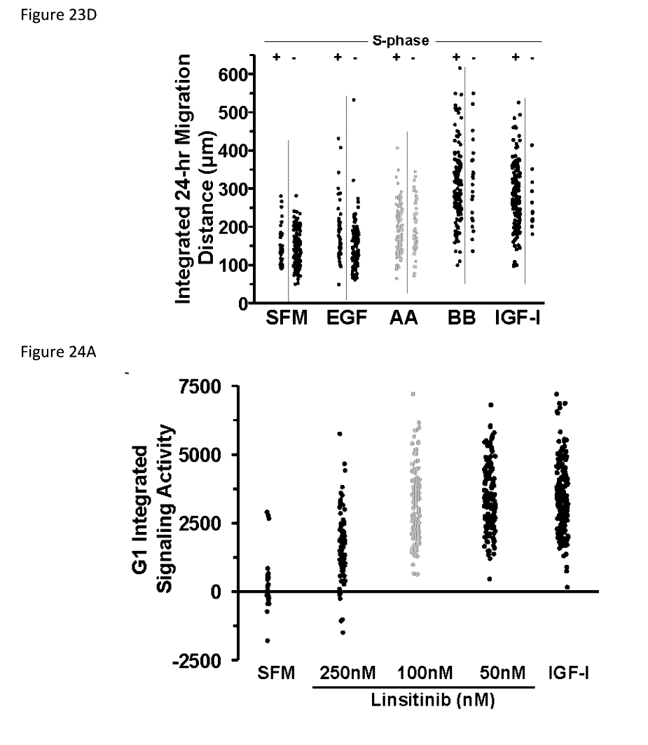

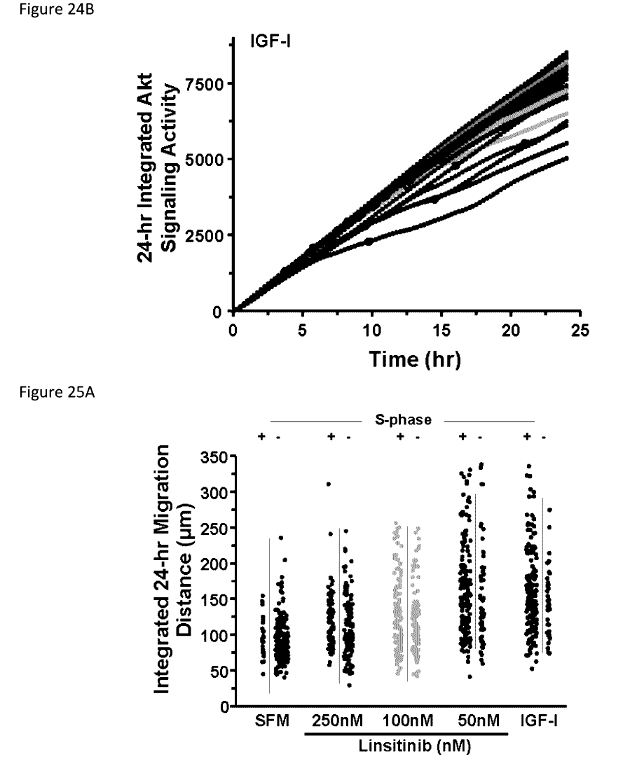

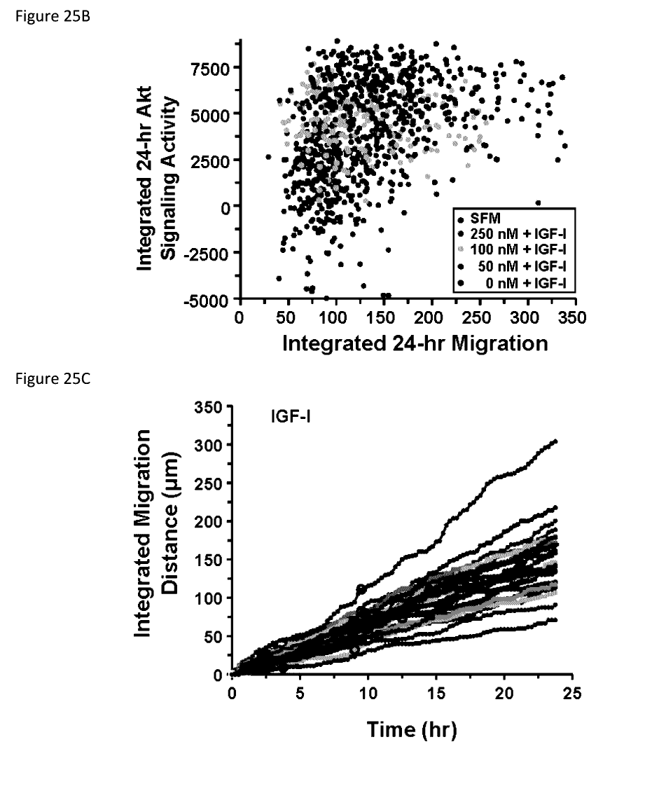

To more broadly assess the dynamics of signaling by different growth factors and their receptors, we also developed a HeLa cell line that stably expresses the FoxO1-clover reporter protein. In HeLa FoxO1-clover cells, IGF-I [500 pM] and insulin [1400 pM] produced sustained signaling effects whereas EGF [4.2 nM] and PDGF-BB [4.1 nM] caused transient responses (FIG. 16A). Treatment with PDGF-AA [3.5 nM] was ineffective, as presumably HeLa cells lack specific receptor expression. Analysis by live-cell imaging of individual cells confirmed the different response patterns observed at the population level (FIGS. 16B-16E). Although there was some variability, IGF-I [and insulin (not shown)] caused sustained effects, while responses to both EGF and PDGF-BB were more transient and heterogeneous (FIGS. 16B-16E). Thus, variation in signaling dynamics among different growth factors, as assessed by live-cell imaging, appears to be a general property that is not unique to a single cell line.

Peptide growth factors influence cellular behavior by engaging trans-membrane receptors and activating a broad range of intracellular signaling responses. Although each growth factor typically binds to a unique receptor, many of the downstream signaling cascades are shared, leading to the question of how different growth factors cause specific behavioral responses. Here we have examined the effects of several growth factors on the PI3-kinase-Akt signaling pathway by using a recently developed sensor composed of a fusion between a modified FoxO1 transcription factor and the green fluorescent protein, clover. The results described in this Example reveal how different growth factors can encode distinct cellular behaviors, and elucidate new information about the dynamics of the PI3-kinase-Akt pathway.

Population Dynamics of Growth Factor Signaling:

Live-cell imaging showed that IGF-I promotes long-term activation of PI3-kinase-Akt signaling in cultured fibroblasts and myoblasts. Exposure of cells to IGF-I led to sustained Akt signaling responses that showed dose-dependent increases in magnitude, as measured by the fraction of the fluorescent FoxO1-clover reporter protein trans-located from the nucleus to the cytoplasm. At the population level, more cytoplasmic localization of the reporter correlated with more Akt phosphorylation and with increased phosphorylation of another Akt substrate as seen by immunoblotting. These results prompted us to investigate how other growth factors encode their tyrosine kinase receptors into Akt signaling dynamics.

Different growth factors induce distinct patterns of Akt activity. Like IGF-I, exposure of cells to insulin led to sustained Akt signaling, with concentration-dependent increases in the fraction of the FoxO1-clover sensor trans-located out of the nucleus (FIG. 9C). By contrast, incubation of cells with EGF promoted more transient signaling, as evidenced by an early dose-dependent peak of reporter accumulation in the cytoplasm, followed by a gradual decline back toward baseline levels (FIG. 10A). These latter results agree with some previous observations, in which Akt was transiently activated by EGF, although in many other studies in multiple cell types, Akt signaling was maintained for long durations after EGF treatment.

Exposure of cells to PDGF-AA or PDGF-BB led to more complex signaling patterns. At low PDGF-BB concentrations, population responses resembled those seen with the highest levels of PDGF-AA, with an initial rapid peak of cytoplasmic translocation of the Akt reporter followed by a gradual return to the nucleus (FIG. 13A). These results were not caused by rapid degradation of growth factor in the extracellular environment, as the same transient response was recapitulated in the presence of high PDGF-BB concentrations with small molecule pathway inhibition (FIGS. 15A, 15B). Similarly, there did not appear to be preferential activation of the PDGF-α receptor by PDGF-BB, as specific receptor inhibition by an antibody had minimal effects on signaling dynamics (FIGS. 14A-14D). At higher concentrations of PDGF-BB, Akt activity was more sustained (FIG. 13A), with signaling resembling the patterns of insulin or IGF-I. Previous studies in 3T3 fibroblasts tracking Akt phosphorylation by serial immunoblotting showed similar dose-dependent results, but did not note the complicated signaling dynamics that observed herein using live-cell imaging. PDGF signaling responses also differed in HeLa cells, as PDGF-AA was ineffective, and PDGF-BB produced only transient responses (FIG. 16A), indicating that other factors, such as the total number of receptors, significantly influences PDGF-BB signaling dynamics.

Growth Factor Signaling Dynamics in Individual Cells:

For all growth factors tested, responses in individual cells varied dramatically, with some cells showing rapid and maximal redistribution of the FoxO1-clover reporter protein from the nucleus to the cytoplasm after growth factor exposure, and others responding minimally. For insulin and PDGF-BB [and IGF-I], single cells in the population yielded more consistent and extensive responses after incubation with higher growth factor concentrations. This was not true for EGF or PDGF-AA, where signaling was highly heterogeneous regardless of growth factor dose. Collectively, this work shows that population averages provide a poor measure of single cell behavior, and illustrate an important advantage of live-cell imaging over the more static measurements of end-point assays, as the former approach makes it possible to capture the full range of cellular signaling activity.

Although these findings demonstrate that Akt signaling dynamics are highly variable among the cells in a population, they also illustrate some fundamental similarities. First, in all of our experiments, Akt activity appears to be encoded in an analog pattern, with different growth factors promoting dose-dependent changes in the peak response, rather than signaling in an all or-none, or digital manner. Second, we found that signaling latency was consistent among different growth factors, with the initial subcellular relocation of the FoxO1-clover reporter being measured within ^{˜}2 min after growth factor addition to cells. Third, the half-maximal peak response to the highest growth factor concentration was recorded at a similar time, within ^{˜}6±2 min after initial exposure. Thus, tracking signaling activity from multiple growth factors by live-cell imaging in the same cellular background can reveal commonalities of signaling patterns as well as unique differences.

Wiring of Receptor—Akt Interactions:

Exposure of cells to IGF-I produced sustained activation of Akt, but EGF induced only transient signaling (FIGS. 10A-10E). Sequential incubation of cells with EGF yielded a minimal second response, but prior exposure to EGF did not block full Akt activation by IGF-I (FIG. 10G). Thus, negative feedback for EGF-mediated signaling probably resides at the level of EGF receptors and not within the downstream PI3-kinase-Akt module. Negative feedback is thus likely to be secondary either to receptor internalization kinetics or dynamics (Goh L K and Sorkin A, Cold Spring Harb Perspect Biol 5, a017459 (2013); incorporated by reference herein) the presence of an inducible receptor inhibitor such as Mig6 (Anastasi S et al, Semin Cell Dev Biol 50, 115-124 (2015); incorporated by reference herein), or other receptor-associated modulatory proteins (Kaushansky A et al, Mol Biosyst 4, 643-653 (2008); incorporated by reference herein). This pattern of limited responsiveness to sequential growth factor treatment observed with EGF also differs from what was observed previously with IGF-I, in which a second growth factor exposure promoted a robust signaling response nearly identical to the initial treatment. This comparison reveals that, depending on the growth factor and receptor, prior signaling history can have anywhere from a large or insignificant impact on subsequent activity.

The application of inhibitors that perturbed different signaling components allowed us also to identify potential wiring principles that govern the relationship between the PI3-kinase-Akt module and different receptor tyrosine kinases. Blockade of both PDGF receptors with the small molecule Sunitinib reduced a sustained maximum response to a dose-dependent transient effect (FIG. 15A), as did inhibition of Akt activation with PI103, thus recapitulating the pattern seen with PDGF-AA or with low concentrations of PDGF-BB (compare FIG. 15B with FIG. 13A). Different results were observed when IGF-I-mediated Akt activity was blocked. Addition of PI103 or the IGF-I receptor kinase inhibitor, Linsitinib, each reduced the magnitude but not the duration of FoxO1-clover reporter translocation in response to IGF-I. Collectively, these observations suggest that regulation of signaling dynamics primarily occurs at the receptor level and not further downstream where shared components are used.

Advantages of Live-Cell Imaging in Understanding Signaling Pathways:

Variable conclusions about population responses to different growth factors have been reached by more traditional end-point assays, including the use of serial immunoblotting or immunocytochemistry to probe for Akt or substrate phosphorylation. Here, by continuous monitoring, a consistent and more robust data set could be collected with less experimental effort and fewer assumptions. Furthermore, aspects of Akt signaling in individual cells not possible with other experimental approaches could be observed. For example, using immunoblots it is difficult to distinguish between analog and digital responses, or to detect differences in peak signaling activity and timing because of the limited temporal and dynamic resolution of this modality. Although immunocytochemical studies can give insights into signaling in individual cells, they require the assumption that different cells are equivalent, and that all cells respond synchronously, which we find is not true. Furthermore, repeated stimuli cannot be tracked using this approach without an inordinately large number of controls, and studies such as those addressing combinatorial growth factor signaling or the effects of different inhibitors on signaling dynamics and kinetics cannot be performed accurately. Thus, experiments using real-time live-cell imaging and a fluorescent reporter can reveal a wealth of signaling information not otherwise attainable.

Implications of Heterogeneous Signaling Dynamics:

Receptor tyrosine kinases typically control multiple downstream signaling cascades. It is likely that the combinatorial interplay of these pathways along with differences in signaling dynamics, as found here, defines the specifics of growth factor actions in different cell types. It thus will be important to develop robust live cell imaging readouts for other signaling modules in order to elucidate the full picture of how growth factor-mediated signaling dynamics are translated into 399 unique cell behaviors, and how these behaviors influence normal physiology and disease. For instance, it can be predicted that brief activation by EGF or PDGF-AA of Akt signaling would not be sufficient to promote cell cycle progression in this model. This would mirror results observed in individual 3T3-L1 cells, in which short-term and low-amplitude stimulation of the PI3-kinase-Akt pathway by PDGF was inadequate to induce translocation of the GLUT4 glucose transporter to the cell membrane (in contrast to the longer and larger effects of insulin). From a more fundamental perspective, comprehensive live-cell imaging studies with multiple readouts should allow a better understanding of the encoding process, and how downstream pathways are controlled in time and space to trigger distinctive cellular responses.

Reagents:

Fetal bovine serum (FBS) was obtained from Hyclone (Logan, Utah). Dulbecco's modified Eagle's medium (DMEM), FluoroBrite, phosphate-buffered saline (PBS), and trypsin/EDTA solution were purchased from Gibco-Life Technologies (Carlsbad, Calif.). Protease inhibitor and NBT/BCIP tablets were from Roche Applied Sciences (Indianapolis, Ind.), and okadaic acid was from Alexis Biochemicals (San Diego, Calif.). Polybrene was purchased from Sigma-Aldrich (St. Louis, Mo.), puromycin was from Enzo Life Sciences (Farmingdale, N.Y.), 6-well tissue culture dishes were from Greiner Bio-One (Monroe, N.C.), and 24-well tissue culture plates were from Corning Inc. (Corning, N.Y.). AquaBlock EIA/WIB solution was from East Coast Biologicals (North Berwick, Me.). The following peptide growth factors were purchased from the listed vendors: R3-IGF-I (GroPep, Adelaide, Australia), recombinant human PDGF-BB (Invitrogen, Carlsbad, Calif.), mouse EGF (Gibco-Life Technologies), recombinant human PDGF437 AA (Thermo Scientific, Rockford, Ill.), and recombinant human insulin (Tocris Bioscience, Bristol, United Kingdom). Peptides were solubilized in 10 mM HCl with 1 mg/ml bovine serum albumin, stored in aliquots at −80° C., and diluted into FluoroBrite imaging medium immediately prior to use. Chemical inhibitors included: Linsitinib (ApexBio, Houston, Tex.), Sunitinib (LC Laboratories, Woburn, Mass.), PI103 (Tocris Bioscience). All inhibitors were solubilized in DMSO, and diluted into imaging medium just prior to use. A neutralizing antibody to the PDGF-α receptor (#AF1062), and an isotype-identical negative control antibody (#AB-108-C) were purchased from R&D Systems (Minneapolis, Minn.). Other primary antibodies included anti-phospho-PRAS40 (Cell Signaling (Beverly, Mass.), catalog #2997), anti-PRAS40 Thr246 (#2691), anti-phospho-AktThr308 (#2965), and anti-Akt (#2691). Secondary antibodies were from Invitrogen (Carlsbad, Calif.), goat anti-rabbit-IgG conjugated to Alexa Fluor 680, and Rockland (Gilbertsville, Pa.), IR800-conjugated goat anti-rabbit IgG. Other reagents and chemicals were purchased from commercial vendors.

Lentiviral Infection and Selection:

HeLa cells (ATCC #CCL2) were incubated in DMEM supplemented with 10% FBS. Cells were transduced at 50% of confluent density with concentrated FoxO1-clover virus in the presence of polybrene (6 μg/ml), as described (Gross S M and Rotwein P, Skelet Muscle 3, doi10.1186/2044-5040-3-10 (2013); incorporated by reference herein), and sorted by fluorescence intensity with a Becton-Dickinson Influx cell sorter. Reporter expression was stable for at least 10 passages in each sorted cell population. C3H10T1/2 mouse embryonic fibroblasts (ATC #CCL226) stably expressing FoxO1-clover (Gross and Rotwein, 2015), were maintained under selection with puromycin (2 μg/ml).

Live Cell Imaging:

Live cell imaging was performed using an EVOS FL Auto microscope with a stage top incubator that was maintained at 37° C. and 95% humidified air with 5% CO₂. Images were collected at 100× magnification at different intervals, using a 10× Fluorite objective (numerical aperture: 0.3), and a GFP light cube (excitation peak, 472/22 nm; emission peak, 510/42 nm). Images were analyzed with the NIH ImageJ plug-in Fiji (NIH, Bethesda, Md.), after using the Polynomial Fit plug-in to subtract background fluorescence, the Stack Reg (rigid registration) plug-in to register images, and the Gaussian Blur plug-in (at 2-pixels) to average fluorescence across pixels. Individual cells were manually tracked using the mTrackJ plug-in (Meijering E et al, Methods Enzymol 504, 183-200 (2012); incorporated by reference herein) by selecting a single point in the nucleus. Cells that died, divided, or migrated out of frame were excluded from analysis. In experiments performed in 6-well dishes, two locations on opposite sides of the well were imaged, while for studies using 24-well plates, one central location was imaged. In each location, at least 25 cells were tracked. The relative nuclear intensity of the FoxO1-clover reporter protein was calculated in each cell by normalizing the values measured at time 0 to 100%. This corresponded to incubation in serum-free medium (SFM). In graphs in which single cell responses were plotted with C3H10T1/2 cells, we applied a time-weighted smoothing filter to each data point. This consisted of averaging contributions from the two prior and two succeeding times (adding 50% of the prior or succeeding time point, and 25% of the next succeeding or earlier time point to 100% of the value of the time point in question, and then dividing by 2.5).

Imaging Protocols:

Short-term responses to individual growth factors: C3H10T1/2 cells were incubated in serum-free Fluorobrite medium plus 2 mM L-glutamine and 0.1% bovine serum albumin for 90 min. Different concentrations of growth factors were added and images were collected every 2 min for 90 min. Growth factors included insulin [0 to 1400 pM], EGF [0 to 4.2 nM], PDGF-AA [0 to 1400 pM], or PDGF-BB [0 to 104 pM]. HeLa cells were incubated in serum-free Fluorobrite medium for 120 min and then incubated with insulin [1400 pM], EGF [4.2 nM], PDGF-AA [3.5 nM], PDGF-BB [4.1 nM], or R3-486 IGF-I [500 pM], with images collected every 2 min.

Sequential growth factor treatments: C3H10T1/2 cells were incubated in serum-free Fluorobrite medium for 90 min, followed by addition of EGF [4.2 nM] or SFM for 60 min. Either EGF [4.2 nM], R3-IGF-I [500 pM], or SFM was added; images were recorded every 2 min for 120 min.

Inhibitor studies: [1] C3H10T1/2 cells were incubated in serum-free Fluorobrite medium containing either anti-PDGF-α antibody or IgG [each at 2.5 μg/ml] for 3 hr, followed by addition of PDGF-AA [1400 pM], or PDGF-BB [10.4 or 83.2 pM]. [2] C3H10T1/2 cells were incubated in serum-free Fluorobrite medium for 60 min, followed by addition of Sunitinib [0 to 100 nM], Linsitinib [0 to 250 nM], or PI103 [0 to 200 nM] for 30 min. Either PDGF-BB [830 pM] or R3-IGF-I [500 pM] was added, and images were collected every 2 min for 90 min. For all imaging studies a minimum of 3 independent experiments were performed.

Protein Extraction and Immunoblotting:

C3H10T1/2 cells stably expressing FoxO1-clover were incubated in SFM with Fluorobrite imaging media for 90 min followed by addition of insulin [1400 pM], EGF [4.2 nM], PDGF-AA [1400 pM], or PDGF BB [104 pM]. Whole protein lysates were collected after 0, 5, 15, 30, 60, and 90 min of growth factor exposure by washing cells twice with cold PBS and addition of RIPA buffer containing protease and phosphatase inhibitors (Mukherjee A and Rotwein P, Mol Endocrinol 22, 1238-1250 (2008); incorporated by reference herein). Protein aliquots (12.5 μg/lane) were resolved by SDS PAGE (10% separating gels), and transferred onto Immobilon-FL membranes. Membranes were incubated in 50% AquaBlock for 60 min, followed by addition of primary antibodies at 1:1000 dilution for 16 hr, and secondary antibodies for 90 min at 1:5000. Images were collected using the LiCoR Odyssey and analysis software version 3.0 (Lincoln, Nebr.).

Receptor Gene Expression:

The relative amount of each receptor mRNA was assessed using RNA seq data (Encode Project Consortium, 2012). In the UCSC mouse genome browser, the Caltech RNA-seq track for C3H10T1/2 cells was chosen, and for each receptor the peak number of unique reads was determined within a 3-exon viewing window.

1. A method of identifying a test compound as an agonist of Akt activity, the method comprising:
providing a first Akt expressing cell, the first Akt expressing cell comprising a biosensor, the biosensor comprising a first polypeptide of SEQ ID NO: 1, SEQ ID NO: 2, SEQ ID NO: 3, or a homolog with at least 95% amino acid identity thereto provided that the homolog has equivalent activity to SEQ ID NO: 1, SEQ ID NO: 2, or SEQ ID NO: 3, and a second polypeptide comprising a fluorescent protein where the second polypeptide is N-terminal or C-terminal relative to the first polypeptide, in a first media, where the first media does not activate Akt;
providing a second Akt expressing cell in the first media, the second Akt expressing cell comprising the biosensor;
contacting the first Akt expressing cell with a first composition comprising a first test compound at a first concentration and a vehicle,
contacting the second Akt expressing cell, with a second composition, the second composition consisting of the vehicle, thereby creating a negative control;
measuring the relative nuclear intensity of the fluorescent protein over time in the first Akt expressing cell;
measuring the relative nuclear intensity of the fluorescent protein over time in the negative control;
where a higher rate of decrease of the relative nuclear intensity of the fluorescent protein in the first Akt expressing cell relative to that of the negative control is an indication that the test compound is an agonist of Akt activity.

2. The method of claim 1 wherein the fluorescent protein is Clover fluorescent protein (SEQ ID NO: 4) or mKate fluorescent protein (SEQ ID NO: 5).

3. The method of claim 1 further comprising providing a third Akt expressing cell, the third Akt expressing cell comprising the biosensor, in the first media and contacting the third Akt expressing cell with a composition comprising the first test compound at a second concentration and the vehicle and calculating a dose-response relationship for the first test compound.

4. The method of claim 1 where the test compound comprises a protein, antibody, or small molecule.

5. The method of claim 1 wherein the cell expresses Akt endogenously.

6. The method of claim 1 further comprising measuring the relative cytoplasmic activity of the fluorescent protein over time in the first Akt expressing cell and in the negative control and where a higher rate of increase of the relative cytoplasmic activity is an indication that the test compound is an agonist of Akt activity.

7. The method of claim 1 where the media is a serum free media.

8. The method of claim 1 where the first Akt expressing cell comprises a first expression vector, the first expression vector comprising a first polynucleotide, the first polynucleotide encoding the biosensor and a promoter operably linked to the first polynucleotide.

9. The method of claim 1 where measuring the relative nuclear intensity comprises live cell imaging.

10. A method of identifying a test compound as an antagonist of Akt activity, the method comprising:
providing a first Akt expressing cell, the first Akt expressing cell comprising a first expression vector, the first expression vector comprising a biosensor, the biosensor comprising SEQ ID NO: 1, SEQ ID NO: 2, SEQ ID NO: 3, or a homolog with at least 95% identity thereto provided that the homolog has equivalent activity to SEQ ID NO: 1, SEQ ID NO: 2, or SEQ ID NO: 3, a second polypeptide encoding a fluorescent protein positioned N or C terminal relative to the first polypeptide in a first media, where the first media comprises a composition that is known to activate Akt;
providing a second Akt expressing cell in the first media, the second Akt expressing cell comprising the first expression vector;
contacting the first Akt expressing cell with a first composition comprising a first test compound at a first concentration in a vehicle,
contacting the second Akt expressing cell with a second composition, the second composition consisting of the vehicle, thereby creating a negative control,
measuring the relative nuclear intensity of the fluorescent protein over time in the first Akt expressing cell;
measuring the relative nuclear intensity of the fluorescent protein over time in the negative control;
where a higher rate of increase of the relative nuclear intensity of the fluorescent protein in the first Akt expressing cell relative to that of the negative control indicates that the test compound is an antagonist of Akt activity.

11. The method of claim 10 wherein the composition that activates Akt comprises IGF-1, fetal bovine serum, insulin, or PDGF-ββ.

12. The method of claim 10 wherein the fluorescent protein is Clover fluorescent protein (SEQ ID NO: 4) or mKate (SEQ ID NO: 5).

13. The method of claim 10 further comprising providing a third Akt expressing cell, the third Akt expressing cell comprising the first expression vector, in the first media and contacting the third Akt expressing cell with a composition comprising the first test compound at a second concentration and the vehicle and calculating a dose-response relationship for the first test compound.

14. The method of claim 10 wherein the test compound comprises a protein, antibody, or small molecule.

15. The method of claim 10 wherein the cell expresses Akt endogenously.

16. The method of claim 10 further comprising measuring the relative cytoplasmic activity of the fluorescent protein over time in the first Akt expressing cell and in the negative control and where a lower rate of increase of the relative cytoplasmic activity of the fluorescent compound indicates that the test compound is an inhibitor of Akt activity.

17. The method of claim 10 where the first Akt expressing cell comprises a first expression vector, the first expression vector comprising a first polynucleotide, the first polynucleotide encoding the biosensor and a promoter operably linked to the first polynucleotide.

18. The method of claim 10 where measuring the relative nuclear intensity comprises live cell imaging.
